(19) United States
(12) Patent Application Publication
(10) Pub. No.: US 2025/0333786 A1
(43) Pub. Date: Oct. 30, 2025

(54) METHODS FOR SINGLE CELL SEQUENCING AND ERROR RATE REDUCTION

(71) Applicant: Nanogami GMBH, Leipzig (DE)

(72) Inventor: Nava Edmond WHITEFORD, Midori-ku, Chiba, Chiba (JP)

(21) Appl. No.: 18/870,145

(22) PCT Filed: Jun. 19, 2023
(86) PCT No.: PCT/US2023/068663

§ 371 Date: Nov. 27, 2024

Related U.S. Application Data
(60) Provisional application No. 63/353,590, filed on Jun. 18, 2022.
(60) Provisional application No. 63/353,591, filed on Jun. 18, 2022.

(51) Int. Cl. C12Q 1/6874

(52) U.S. Cl. CPC C12Q 1/6874

(57) ABSTRACT

The present disclosure in some aspects relates to real-time nucleic acid sequencing, including real-time sequencing, single molecule sequencing, long-read sequencing, and/or single-cell sequencing. Also described herein are methods of error reduction and of analyzing sequencing data obtained from the sequencing methods. In one aspect, provided herein is a method for nucleic acid sequencing, comprising: a) localizing a double stranded nucleic acid to a location on a substrate; b) generating a first single stranded nucleic acid and a second single stranded nucleic acid from the localized double stranded nucleic acid; c) restricting diffusion of the first and/or second single stranded nucleic acids; d) attaching the first and second single stranded nucleic acids at sites near the location on the substrate; e) obtaining sequencing reads from the attached first single stranded nucleic acid and sequencing reads from the attached second single stranded nucleic acid. In some embodiments, the method further comprises: f) associating a sequence of the first single stranded nucleic acid with a sequence of the second single stranded nucleic acid, thereby determining a sequence of the double stranded nucleic acid.

### CROSS-REFERENCE TO RELATED APPLICATIONS

This application claims priority to U.S. Provisional Patent Application No. 63/353,590, filed Jun. 18, 2022, entitled “Methods for Low Cost DNA Sequencing,” and U.S. Provisional Patent Application No. 63/353,591, filed Jun. 18, 2022, entitled “Sample Preparation Methods for Error Rate Reduction and Single Cell Sequencing,” which are herein incorporated by reference in their entireties for all purposes.

### FIELD

The present disclosure generally relates to methods and compositions for determining a sequence of a nucleic acid molecule, including methods and compositions for nucleic acid sequencing such as single cell nucleic acid sequencing, and/or error rate reduction in sequencing of nucleic acids involving double stranded nucleic acids.

### BACKGROUND

The analysis of nucleic acid molecules is an extremely complex endeavor which typically requires accurate, rapid characterization of large numbers of nucleic acid molecules via high throughput DNA sequencing. The determination of nucleic acid sequences remains a laborious and difficult task, particularly in comparison to cheaper probe based methods such as qPCR (also called real-time PCR). Simplifying and reducing the cost of sequencing therefore remains an important problem. The present disclosure addresses these and other needs.

### BRIEF SUMMARY

Known nucleic acid sequencing-by-synthesis (SBS) methods are cyclic and require deactivation of signal from a labeled nucleotide incorporated in one cycle and removal of labeled nucleotides that are not incorporated in that cycle, prior to introducing labeled nucleotides for the next cycle. For example, in some existing methods, dye-labeled “A” nucleotides (e.g., dATP labeled with a first fluorophore) would be introduced into a flow cell, incorporated and detected at particular spots in the flow cell (e.g., indicating a base “T” in the template molecules at those spots), and then the dye in the incorporated nucleotides at those particular spots would be bleached (and unincorporated dye-labeled nucleotides removed from the flow cell) before dye-labeled “T” nucleotides (e.g., dTTP labeled with a second fluorophore that is of a different “color” compared to the first fluorophore) are flowed in the flow cell to interrogate the next base (e.g., base “A” at the 5′ of the base “T” in the template molecules). In a particular cycle, a mixture of dye-labeled nucleotides may be introduced into the flow cell, e.g., four fluorescent dyes each of a different “color” may be used to label A, T, C, and G, respectively (such as in a 4-channel SBS chemistry) or two different fluorescent dyes may be used (e.g., in a 2-channel SBS chemistry using “red” for C, “green” for T, “red” and “green” appearing as “yellow” for A, and unlabeled for G). Regardless, these known SBS methods require deactivation of fluorescent signals, e.g., via cleavage of fluorescently labeled reversible terminators on incorporated nucleotides, in order to allow incorporation of nucleotides to interrogate the next base. One or more washes between flow cell cycles are also performed, e.g., in order to remove unincorporated nucleotides and/or cleaved fluorescent labels. These and other requirements of known SBS methods have kept their costs high (e.g., as compared to methods based on qPCR or antigen-antibody interactions) and have limited their applications, especially in sequencing-based diagnostics, for instance, in response to a pandemic such as COVID-19 where large numbers of samples must be sequenced in a short period of time.

In one aspect, provided herein is a method for nucleic acid sequencing, comprising: a) localizing a double stranded nucleic acid to a location on a substrate; b) generating a first single stranded nucleic acid and a second single stranded nucleic acid from the localized double stranded nucleic acid; c) restricting diffusion of the first and/or second single stranded nucleic acids; d) attaching the first and second single stranded nucleic acids at sites near the location on the substrate; e) obtaining sequencing reads from the attached first single stranded nucleic acid and sequencing reads from the attached second single stranded nucleic acid. In some embodiments, the method further comprises: f) associating a sequence of the first single stranded nucleic acid with a sequence of the second single stranded nucleic acid, thereby determining a sequence of the double stranded nucleic acid.

In any of the preceding embodiments, the method can comprise allowing or promoting diffusion of the first and/or second single stranded nucleic acids, before restricting diffusion of the first and/or second single stranded nucleic acids.

In any of the preceding embodiments, the localizing in a) can comprise non-covalently attaching the double stranded nucleic acid to the location on the substrate. In any of the preceding embodiments, the double stranded nucleic acid may but does not have to be covalently attached to the substrate or a molecule immobilized thereon. In any of the preceding embodiments, it can be that only one strand of the double stranded nucleic acid is covalently attached to the substrate or a molecule immobilized thereon.

In any of the preceding embodiments, the localizing in a) can comprise attracting and/or confining the double stranded nucleic acid to the location on the substrate using an electrode. In any of the preceding embodiments, the electrode can be integrated in the substrate or separately provided from the substrate. In any of the preceding embodiments, the electrode can be removable from the substrate.

In any of the preceding embodiments, the localizing in a) can comprise hybridizing a region of the double stranded nucleic acid to a nucleic acid probe immobilized directly or indirectly on the substrate. In any of the preceding embodiments, the double stranded nucleic acid can comprise a single-stranded region. In any of the preceding embodiments, the single-stranded region can be a loop, a bulge, or generated by partially melting the double stranded nucleic acid.

In any of the preceding embodiments, the double stranded nucleic acid can be from a cell or tissue sample. In any of the preceding embodiments, the double stranded nucleic acid can be a fragmented DNA. In any of the preceding embodiments, the double stranded nucleic acid can be cell-free DNA or generated by fragmenting genomic DNA. In any of the preceding embodiments, the double stranded nucleic acid can be an amplification product of a cellular DNA or RNA or cell-free DNA. In any of the preceding embodiments, the double stranded nucleic acid can be from a single cell and spaced on the substrate from double stranded nucleic acids from other cells.

In any of the preceding embodiments, the location can be a random location on the substrate. In any of the preceding embodiments, the location can be among locations of an ordered pattern on the substrate. In any of the preceding embodiments, the location can be in a protrusion or an indentation at the location on the substrate. In any of the preceding embodiments, the location can be on a bead at the location on the substrate.

In any of the preceding embodiments, the first and second single stranded nucleic acids can be generated in b) by melting the localized double stranded nucleic acid using heat, change in pH, a denaturing buffer, or any combination thereof.

In any of the preceding embodiments, restricting diffusion of the first and second single stranded nucleic acids in c) can comprise capturing the first and second single stranded nucleic acids by nucleic acid probes immobilized directly or indirectly on the substrate. In any of the preceding embodiments, the nucleic acid probes can be at sites near the location on the substrate.

In any of the preceding embodiments, restricting diffusion of the first and second single stranded nucleic acids in c) can comprise confining the first and second single stranded nucleic acids using an electrode. In any of the preceding embodiments, the electrode can be integrated in the substrate or separately provided from the substrate. In any of the preceding embodiments, the electrode can be removable from the substrate.

In any of the preceding embodiments, in d), the first and second single stranded nucleic acids independently can be covalently and/or noncovalently attached at the sites near the location on the substrate. In some embodiments, in a), only one of the first and second strands of the double stranded nucleic acid is covalently immobilized to the location on the substrate, and in b), the other strand is separated from the immobilized strand and allowed to diffuse, and its diffusion is restricted in c) and it is attached to a site near the location in d). In some embodiments, in a), both strands of the double stranded nucleic acid are noncovalently localized to the location on the substrate, and in b), the strands are separated from each other and allowed to diffuse, and their diffusion is restricted in c) and both strands can be independently attached to sites near the location in d).

In any of the preceding embodiments, in d), the sites may be no more than about 8 μm, no more than about 6 μm, no more than about 4 μm, no more than about 2 μm, no more than about 1 μm, no more than about 0.5 μm, or no more than about 0.25 μm from the location. In any of the preceding embodiments, one of sites can be at the location, and the other site can be no more than about 2 μm, no more than about 1 μm, no more than about 0.5 μm, or no more than about 0.25 μm from the location. In any of the preceding embodiments, both sites may be no more than about 2 μm, no more than about 1 μm, no more than about 0.5 μm, or no more than about 0.25 μm from the location.

In any of the preceding embodiments, the attached first single stranded nucleic acid and the attached second single stranded nucleic acid may be amplified on the substrate. In any of the preceding embodiments, the attached first single stranded nucleic acid and the attached second single stranded nucleic acid may be clonally amplified to form clusters of amplicons on the substrate, and be sequenced using a cluster-based sequencing method disclosed herein. In any of the preceding embodiments, the attached first single stranded nucleic acid and the attached second single stranded nucleic acid may be amplified using bridge amplification.

In any of the preceding embodiments, the attached first single stranded nucleic acid and the attached second single stranded nucleic acid may but do not have to be amplified or can be only minimally amplified on the substrate. In any of the preceding embodiments, in e), the sequencing reads can be obtained using single molecule sequencing. In any of the preceding embodiments, in e), the sequencing reads can be obtained using real-time sequencing, optionally single molecule real-time sequencing.

In any of the preceding embodiments, in e), the sequencing reads can be obtained using sequencing-by-synthesis, sequencing-by-binding, avidity sequencing, sequencing-by-ligation, and/or sequencing-by-hybridization. In any of the preceding embodiments, in e), the sequencing reads can be obtained by imaging the substrate and recording optical signals in sequential cycles of imaging at each of the sites.

In any of the preceding embodiments, optical signals at one of the sites can be optically resolvable from optical signals at the other site.

In any of the preceding embodiments, the method can comprise determining the sequence of the first single stranded nucleic acid by comparing multiple sequencing reads from the attached first single stranded nucleic acid, optionally the method comprises aligning the multiple sequencing reads and/or generating a consensus sequence of the multiple sequencing reads.

In any of the preceding embodiments, the method can comprise determining the sequence of the second single stranded nucleic acid by comparing multiple sequencing reads from the attached second single stranded nucleic acid, optionally the method comprises aligning the multiple sequencing reads and/or generating a consensus sequence of the multiple sequencing reads.

In any of the preceding embodiments, the sequence of the first single stranded nucleic acid and the sequence of the second single stranded nucleic acid can be determined independently of one another.

In any of the preceding embodiments, the method can comprise comparing the sequence of the first single stranded nucleic acid and the complement of the sequence of the second single stranded nucleic acid, and/or comparing the sequence of the second single stranded nucleic acid and the complement of the sequence of the first single stranded nucleic acid.

In any of the preceding embodiments, the method can comprise comparing a single-stranded consensus sequence of the first single stranded nucleic acid with a single-stranded consensus sequence of the second single stranded nucleic acid to generate a duplex consensus sequence, optionally wherein one or more errors in sequence are identified using comparison of the single-stranded consensus sequences.

In any of the preceding embodiments, the method can comprise identifying an overlapping sequence between the sequence of the first single stranded nucleic acid and the complement of the sequence of the second single stranded nucleic acid, and identifying a first non-overlapping sequence in the sequence of the first single stranded nucleic acid and/or a second non-overlapping sequence in the complement of the sequence of the second single stranded nucleic acid.

In any of the preceding embodiments, the method can comprise identifying an overlapping sequence between the sequence of the second single stranded nucleic acid and the complement of the sequence of the first single stranded nucleic acid, and identifying a first non-overlapping sequence in the sequence of the second single stranded nucleic acid and/or a second non-overlapping sequence in the complement of the sequence of the first single stranded nucleic acid.

In any of the preceding embodiments, the method can comprise assembling i) the sequence of the first single stranded nucleic acid and the complement of the sequence of the second single stranded nucleic acid, and/or ii) the sequence of the second single stranded nucleic acid and the complement of the sequence of the first single stranded nucleic acid, into a longer sequence than the sequences of the first and second single stranded nucleic acids.

In any of the preceding embodiments, the method can comprise associating the sequence of the first single stranded nucleic acid with the sequence of the second single stranded nucleic acid during basecalling.

In any of the preceding embodiments, the method can comprise associating the sequence of the first single stranded nucleic acid with the sequence of the second single stranded nucleic acid post-basecalling.

In any of the preceding embodiments, the method can comprise determining that the sequence of the first single stranded nucleic acid and the sequence of the second single stranded nucleic acid are derived from the two strands of the same double stranded nucleic acid localized to the substrate in a).

In one aspect, provided herein is a method for nucleic acid sequencing. comprising: a) localizing a single cell or nucleus to a location on a substrate; b) releasing a nuclei acid from the localized single cell or nucleus; c) restricting diffusion of the nucleic acid; d) attaching the nucleic acid at a site at or near the location on the substrate; and e) obtaining sequencing reads from the attached nucleic acid, thereby determining a sequence of the nucleic acid. In some embodiments, the nuclei acid from the localized single cell or nucleus can be a double stranded nucleic acid, or can be used to generate a double stranded nucleic acid localized on the substrate. The double stranded nucleic acid can be analyzed using a method for nucleic acid sequencing according to any of the preceding embodiments.

In any of the embodiments herein, the nucleic acid molecule can comprise a deoxyribonucleotide or derivative or analog thereof and/or a ribonucleotide or derivative or analog thereof. In any of the embodiments herein, the nucleic acid molecule can comprise DNA or RNA. In any of the embodiments herein, a method disclosed herein can be used for direct RNA sequencing without first converting RNA to DNA such as cDNA.

In any of the embodiments herein, the polymerase can be a DNA-dependent polymerase and/or an RNA-dependent polymerase. In any of the embodiments herein, the same polymerase can be used to catalyze multiple nucleotide incorporation events using the same nucleic acid molecule as template. In any of the embodiments herein, the same polymerase can be used to catalyze multiple nucleotide incorporation events using different nucleic acid molecules as template, and the different nucleic acid molecules may be provided on substrate for nucleic acid sequencing. In any of the embodiments herein, different polymerases can be used to catalyze two or more nucleotide incorporation events using the same nucleic acid molecule as template. In any of the embodiments herein, different polymerases can be used to catalyze two or more nucleotide incorporation events using different nucleic acid molecules as template, and the different nucleic acid molecules may be provided on substrate for nucleic acid sequencing. In any of the embodiments herein, the rate(s) of nucleotide incorporation by the one or more polymerases can be controlled.

In any of the embodiments herein, the one or more polymerases can comprise a DNA polymerase and/or an RNA polymerase. In any of the embodiments herein, the polymerase can have a DNA-dependent DNA polymerase activity and/or an RNA-dependent DNA polymerase activity. In any of the embodiments herein, the one or more polymerases can be selected from the group consisting of DNA polymerase I, Klenow fragment of DNA polymerase I, DNA polymerase III, Taq polymerase, KlenTaq polymerase, TopoTaq polymerase, Bst polymerase, rBST DNA polymerase, Bsu polymerase, T7 DNA polymerase, T7 RNA polymerase, T3 DNA polymerase, T3 RNA polymerase, T4 polymerase, T5 polymerase, φ29 polymerase, 9°N polymerase, KOD polymerase, Pfu DNA polymerase, Vent DNA polymerase, Deep Vent DNA polymerase, Vent (exo-) polymerase, M2 polymerase, B103 polymerase, GA-1 polymerase, φPRD1 polymerase, N29 DNA polymerase, SP6 RNA polymerase, a reverse transcriptase (optionally a SuperScript® III reverse transcriptase), and a variant or derivative thereof.

### BRIEF DESCRIPTION OF THE DRAWINGS

The drawings illustrate some embodiments of the features and advantages of this disclosure. These embodiments are not intended to limit the scope of the appended claims in any manner.

FIGS. 1 show a schematic of double stranded fragments of nucleic acid are attached, made single stranded, and clustered for cluster-based sequencing. For instance, a ds DNA can be melted, and one or both of the resulting ssDNA strands can be localized, allowing the source dsDNA sequence to be reconstructed algorithmically.

FIG. 2 shows an exemplary charge/field barrier configuration with reagents in regions marked “A”, “T”, “G”, and “C”, representing adenine, guanine, thymine, and cytosine nucleotides, respectively. Rectangles at the top of the regions with reagents and at the bottom of main chamber (e.g., in which an apyrase can be provided) represent electrodes, where the electrodes in at the top of the regions with nucleotides may be positively charged, e.g., to attract the “A”, “T”, “G”, and “C” nucleotides, confining the negatively charged nucleotides to their respective regions. A nucleotide may be released and/or drawn into the main chamber by switching the electrode within the respective region to be negatively charged, and the electrode in the main chamber can be positively charged with respect to this example.

FIG. 3 shows an exemplary chamber where reagents are confined to vesicles (“reagent bubbles”), represented as circles in the diagram. Regents may be released by breaking the vesicles (e.g., using any one or a combination of the methods disclosed herein, such as charge, heat, change in pH, surfactant, etc.) to then go into the main chamber.

FIG. 4 shows photobleaching and step counting can be used to determine the number of steps observed and the homopolymer length. Step counting, including but not limited to the exemplary algorithm from Shu et al. (“Counting of six pRNAs of phi29 DNA-packaging motor with customized single-molecule dual-view system.” EMBO J. 2007 Jan. 24; 26(2): 527-537 which is herein incorporated by reference in its entirety for all purposes) and Mira et al. (“Counting the Number of Fluorophores Labeled in Biomolecules by Observing the Fluorescence-Intensity Transient of a Single Molecule,” BCSJ 78(9): 1612-18 (2005), which is herein incorporated by reference in its entirety for all purposes), may be used to determine the number of steps observed, and therefore the likely homopolymer length.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods relating to nucleic acid (e.g., DNA/RNA) sequencing in respect to improving the error rate on reads generated from single or double stranded fragments of nucleic acid (e.g., dsDNA). Also provided herein are methods of isolating sequencing reads from single cells.

### A. Error Rate Reduction and Long Read Generation

In some embodiments, provided herein is a method for double stranded nucleic acid sequencing, comprising: a) weakly attaching a double stranded nucleic acid from a sample to a surface; b) melting or denaturing the double stranded nucleic acid to become single stranded, generating a first single stranded nucleic acid and a second single stranded nucleic acid, which are complements of one another and may no longer be attached to the surface, c) restricting diffusion of the first single stranded nucleic acid and the second single stranded nucleic acid from one another and attaching them to the surface, wherein the restricting diffusion of the first single stranded nucleic acid and the second single stranded nucleic acid leads to either one or both of the strands being attached near one another on the surface, d) obtaining spatially localized reads of the first single stranded nucleic acid and the second single stranded nucleic acid, wherein due to the limited diffusion of step c), the first single stranded nucleic acid and the second single stranded nucleic acid can be identified as belonging to the same double stranded nucleic acid due to their proximity on the surface and their reads are combined to improve accuracy.

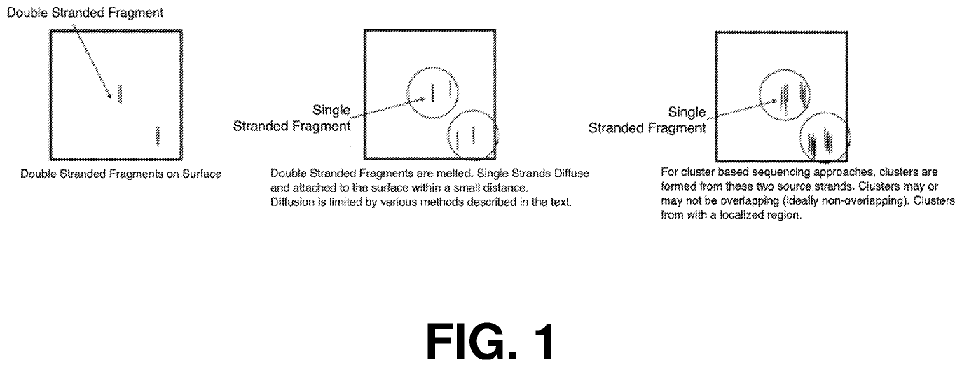

In some embodiments, a double stranded nucleic acid (for example as sourced from circulating free DNA) is weakly attached to a surface. In some embodiments, this surface attachment may use one of a number of methods. For example, the surface may have a (weak) positive charge which attracts the double stranded nucleic acid. In some embodiments, the surface may have embedded electrodes which attract and/or attach to the double stranded nucleic acid. In some embodiments, the DNA may be partially melted and attach to probes (random or otherwise) on the surface (see, e.g., FIG. 1). In some examples, a double stranded fragment is attached on a surface, and are melted. Single strands can diffuse and attach to the surface within a small distance, and the diffusion can be limited by various methods described herein. For cluster based sequencing methods, clusters can be formed from one or both two strands generated from the double stranded fragment, and the clusters may or may not be overlapping. In preferred embodiments, the clusters are not overlapping. In some embodiments, clusters are formed with a localized region.

In any of the embodiments herein, once attached to the surface the double stranded nucleic acid may be melted/denatured (e.g., using heat, or using changes in pH, buffer, or any combination thereof) to form single stranded nucleic acid. In some embodiments, the surface is prepared with probes such that the single stranded nucleic acid attaches to these probes (e.g., as performed in cluster based sequencing-by-synthesis, U.S. Pat. No. 10,370,652 B2 which is incorporated herein by reference in its entirety for all purposes).

In any of the embodiments herein, “probe-less” methods of attachment may be used, for example, charge based attachment. In any of the embodiments herein, the surface may be prepared such that the single stranded nucleic acid(s) do not diffuse widely before attaching to probes or the surface. In any of the embodiments herein, diffusion of the single stranded nucleic acid(s) may be restricted through the use of electric fields. In particular embodiments, the electric field attracts the single stranded nucleic acid(s) toward the surface. In some embodiments, the electric fields may be structured with electrodes, to create barriers preventing the single stranded nucleic acid from diffusing widely. For example, negatively charged electrodes may be used to confine the single stranded nucleic acid(s).

In any of the embodiments herein, once the nucleic acid is attached clusters may be formed (for example through bridge amplification), and sequencing may proceed, as in sequencing-by-synthesis or similar methods.

In some embodiments, the double stranded nucleic acids are randomly attached to the surface. In some embodiments, the double stranded nucleic acids are not randomly attached to the surface, and the surface may be pattern to allow formation of an ordered array of double stranded nucleic acids attached directly or indirectly to the surface. In some embodiments, the surface is a patterned flowcell surface. In some embodiments, the surface can comprise protrusions and/or indentations, such as pillars and/or wells.

In some embodiments, a bead based cluster generation approach can be used, comprising cluster amplification on beads which are then loaded into an unstructured flowcell creating a 3D array of beads which can be imaged by confocal microscopy. Exemplary methods include those described in US 2021/0040555 which is incorporated herein by reference in its entirety for all purposes.

In some embodiments, a bead based cluster generation approach can be used, comprising loading beads onto a patterned flowcell comprising two or more wells, wherein each well is separated by about 0.2 μm to about 2.0 μm from any adjacent well and each well comprises at least one particle, said particle comprising a plurality of oligonucleotide moieties covalently attached to said particle via a bioconjugate linker, wherein the bioconjugate linker is formed via a reaction between a particle polymer comprising a first bioconjugate reactive moiety and an oligonucleotide comprising a second bioconjugate reactive moiety, and wherein the average longest dimension of the particle is from about 100 nm to about 1000 nm, wherein said solid support comprises a polymer layer. Clusters are then grown on these beads and imaged fluorescently. Exemplary methods include those described in U.S. Pat. No. 11,629,380 B2, which is incorporated herein by reference in its entirety for all purposes.

In any of the embodiments herein, during image analysis sequence, signals at the multiple locations on the surface are recorded and this may use super resolution to obtain precise localization. In some embodiments, post-basecalling or as part of the basecalling process an algorithm is used to determine if two nearby templates (e.g., using Euclidean distance or otherwise) came from the same source fragment. In some embodiments, determining if two nearby templates came from the same source fragment is based on their proximity. In some embodiments, determining if two nearby templates came from the same source is used as long as the read length is long enough to result in an overlap between the forward and reverse strand. In some embodiments, if two strands are determined to come from the same source double stranded nucleic acid, information from these two strands maybe combined.

In some embodiments, a method disclosed herein can comprise masking bases (e.g., replacing them with “N”s) that do not match when the strand sequences are converted into the same orientation. In some embodiments, base quality scores (as determined during basecalling) may be used to select the most accurate basecall. In some embodiments, features extracted during image analysis may provide additional information to inform this process (for example circularity of the cluster in the position of a likely errored position, local background, or other errors).

In some embodiments, sequencing-by-synthesis may be used and errors in each strand are likely to have different error characteristics. In some embodiments, this is due to the nature of phasing errors in these approaches. In some embodiments, early bases (e.g., bases towards the 3′ end of a sequencing template) are likely be of higher quality and can be used in preference to later bases in the complementary strand. In some embodiments, determining if two nearby templates came from the same source fragment can be used to facilitate basecalling, for example, to achieve more accurate basecalling.

In some embodiments, a number of algorithmic approaches may be used to combine the above information either during or post-basecalling. In some embodiments, determining if two nearby templates came from the same source fragment can increase effective read length, where strands are longer than the platform read length but short enough that an overlap exists. In some embodiments, the overlap may be used to locally assemble two nearby reads into a longer read.

In some embodiments, determining if two nearby templates came from the same source fragment is used for clusters that are well separated. In some embodiments, it may be possible to use information from clusters formed from both forward and reverse strands (which would normally be termed mixed, or mixed template clusters). In some embodiment, an HMM or other algorithm may be used to extract the most likely source template from the convolved signal of the forward and reverse strands.

### B. Single Cell and Spatial Sequencing

In some embodiments, isolating and sequencing single cells is of interest for a number of applications. In any of the embodiments described herein, single cells are attached to a surface. In some embodiments, the attachment may be a charge-based attachment, or a binding attachment using a monoclonal or polyclonal mix of antibodies.

In some embodiments, attachment sites may be spread across a surface, either randomly or in a pattern. In some embodiments patterned attachment sites may be of a size such that only a single cell can attach to a single attachment site.

In some embodiments, once attached to the surface, cells may be lysed (e.g., using SDS or other lysis buffer). In some embodiments, other methods maybe used to break the cell bilayer to lyse cells (e.g., toxins, heat, pH etc.). In some embodiments the attachment site may include surface attached (or otherwise) reverse transcriptase. In some embodiments, the reverse transcriptase can be used to convert cellular RNA to DNA.

In some embodiments, charge barriers around the cell may be used to confine extracted material to regions near the location of the cell (e.g., the attachment site) on a surface to which the cell was previously attached. In some embodiments, charge barriers may include negatively charged patterned electrodes which repel the negative charged DNA/RNA and prevent it from diffusing from the location of the cell. In some embodiments, a positive electrode may also be used to pull the DNA/RNA down on the attachment site.

In some embodiments, the DNA/RNA may then be confined to the surface, for example, either through a charge based interaction, or the use of probes (e.g., random probes, a specific ligated adapter, polyT probes to attach to introduced polyA tails, or targeted probes). The polyA tails can be those in mRNA molecules, or a polyA sequence can be introduced to attach to a DNA molecule.

In some embodiments, index sequences may then be introduced to the confined fragments. In some embodiments, indexes may be introduced using known methods, such as ligation or PCR based methods. In some embodiments, indexes may have been patterned on the surface, and released using a number of methods (e.g., optically cleaved or otherwise released using heat/charge-based methods) including established methods.

In some embodiments, any one or more of the above methods may be used in a spatial sequencing context. In particular embodiments, cells maintain orientation from the source tissue, and nucleic acid molecules (e.g., cellular DNA/RNA, probes for cellular DNA/RNA, or ligation and/or amplification products of the probes or the cellular DNA/RNA) can be migrated from the cells or tissue sample and be captured at corresponding locations on an artificial substrate, e.g., by capture probes immobilized on the artificial substrate.

The present disclosure in some aspects relates to methods and systems for determining the nucleotide sequence of individual nucleic acid molecules using optical techniques. In some embodiments, disclosed herein are methods for imaging labeled nucleotides added onto a nucleic acid molecule mounted on a substrate, e.g., a solid surface, wherein the nucleic acid molecules is sequenced using sequencing-by-synthesis (SBS). Any one or more of the labeled nucleotides can be labeled with only one kind of label (e.g., a fluorophore appearing as “red” or “green”), and may be labeled with one or more molecules of the same label. Further, any one or more of the labeled nucleotides can be labeled with two or more kinds of labels (e.g., a “red” first fluorophore and a “green” second fluorophore such that the labeled nucleotide appears as “yellow”), and may be labeled with one or more molecules of each kind of label. For a multiply labeled nucleotide, the ratio of different kinds of labels can be tuned as needed, e.g., such that labeled nucleotides having different ratios of distinct labels may be distinguished.

The DNA sequencing method most commonly used until the 2000s was dideoxy chain terminator sequencing (Sanger et al., PNAS 74(12): 5463-5467 (1977), incorporated herein by reference in its entirety for all purposes). However, this method is time-consuming, labor-intensive, expensive, and low throughput. To overcome some of these deficiencies massively parallel sequencing (MPS) approaches were developed by Solexa and others (e.g., U.S. Pat. No. 7,115,400 incorporated herein by reference in its entirety for all purposes). These approaches sequence large numbers of nucleic acids in parallel, and have drastically reduced cost-per-base.

However, key deficiencies remain. In particular, while MPS has reduced the cost per base, the cost per run remains high (>$400). Common MPS approaches also work using clusters of amplified DNA. As such they are limited to DNA sequencing, and cannot be used to directly sequence RNA.

Single molecule sequencing (e.g., as implemented by Pacific Biosciences, Helicos and others) addresses some of these issues. However, these approaches have not resulted in lower run cost. In single molecule SBS, photobleaching has been proposed as a method of deactivating labeled nucleotides to avoid signal accumulation (Braslavsky et al.). The counting of discrete bleaching events has been proposed as a method of resolving multiple incorporations (e.g., U.S. Pat. No. 6,221,592 incorporated herein by reference in its entirety for all purposes). In these methods, incorporated dyes are bleached to prevent signal accumulation, since residual signals from previous cycles would interfere with detection in later cycles. Photobleaching must be taken to completion to remove all dye labels before labeled nucleotides are added to start a new cycle.

By bleaching to completion, the existing SBS methods can only be used in a cyclic sequencing context. In addition to this, bleaching to completion exposes the nucleic acid strand to light which may case photo-damage to the nucleic acid before sequencing is complete. It is therefore desirable to minimize illumination of the strands being sequenced. In addition to this, it is not obvious how photo-bleaching would be used in a real-time sequencing context, where nucleotides are continuously and not cyclically introduced.

The present disclosure in some aspects relates to nucleic acid sequencing methods where dye deactivation (for example by photobleaching) limits signal accumulation but is not generally taken to completion prior to incorporation of additional labeled nucleotides in a given strand being sequenced. In some embodiments, a drop in signal intensity (e.g., emission) resulting from dye deactivation may be used to infer information about the strand under synthesis (and the complementary template strand), as part of a nucleic acid sequencing approach. It should be noted that photobleaching and/or any other suitable method of dye deactivation may be used. Exemplary photobleaching techniques are described, e.g., in Chen et al., Mol Biol Cell, 25(22): 3630-42 (2014), incorporated herein by reference in its entirety for all purposes.

In some embodiments, provided herein is a sequencing-by-synthesis method where nucleic acid strands are attached to a solid surface and then extended by a polymerase (e.g., by a DNA polymerase or a reverse transcriptase) to incorporate a nucleic acid molecule (e.g., a nucleotide) comprising a fluorescent (or otherwise emitting) label to the 3′ terminus of a sequencing primer hybridized to a nucleic acid strand.

In some embodiments, an imaging platform capable of resolving single dyes at multiple locations on a substrate is used to image the dyes, and determine the “intensity” of a nucleic acid “spot.” In some embodiments, the term “intensity” used herein refers to a value computed from dye emissions of a single nucleic acid imaged as a “spot.” In some embodiments, the intensity may comprise emissions from one or more molecules of the same dye or different dyes, and may be corrected, for example to compensate for background signal such as background illumination (e.g., background fluorescence, such as autofluoresence). In some embodiments, the imaging system can be used to determine when labels are incorporated (which results in increases in intensity), and when bleaching events have occurred (which results in decreases in intensity).

In some embodiments, by algorithmically combining information regarding incorporation events (intensity increases) and photobleaching events (intensity decreases), the sequence of a single nucleic acid strand can be probabilistically determined. Such an approach is simpler than current sequencing approaches which require multiple reagent cycles, and does not require a nano-fabricated surface.

In some embodiments, disclosed herein are methods where photobleaching is not taken to completion during a single incorporation/imaging cycle. In some embodiments, stepwise increases in signal intensity are used to register the incorporation of labeled nucleotides. In some embodiments, photobleaching steps are used to provide information to determine not just the number of incorporations, but the nucleotide sequence of a strand under synthesis. In some embodiments, multiple labels can be used, where the labeled nucleotides can be distinguished from one another based on the type and/or number of label(s) on an individual labeled nucleotide. These labels may emit at a specific wavelengths, or when filtered, produce a characteristic increase in signal intensity. In some embodiments, a nucleotide incorporation event and a signal deactivation event of the incorporated nucleotide can be matched or paired. For instance, a label that produces a characteristic increase in signal intensity can result in a corresponding characteristic decrease in signal intensity when the label is bleached. In some embodiments, between two time points or by comparing signal intensities between two detection time windows, a change in registered intensity may reflect the type of labeled nucleotide incorporated and be used to determine the complementary sequence in the strand being sequenced.

In some embodiments, labeled nucleotides may but do not need to be added cyclically. In some embodiments, a method disclosed herein may comprise one or more cycles in which one or more labeled nucleotides are added, signals associated with nucleotide incorporations are detected, signals of the incorporated nucleotides are deactivated, and the substrate is washed to remove labeled nucleotides and optionally cleaved labels, before additional labeled nucleotides are added to sequence the next base. In cases where one or more labeled nucleotides are added cyclically, a single label may be used to label the one or more labeled nucleotides in a cycle, for example, similar to a 2-channel SBS chemistry using “red” for C, “green” for T, “red” and “green” appearing as “yellow” for A, and unlabeled for G. In some embodiments, a method disclosed herein may comprise using a single label and introducing labeled nucleotides in one or more cycles, where in each cycle or flow only labeled nucleotides comprising one nucleotide type (e.g., A, T, C, or G) and the single label are introduced in the sequencing reaction, and nucleotide incorporation/non-incorporation is monitored in the one or more cycles. In these embodiments, nucleotides introduced in one cycle are either signal-deactivated (if incorporated) or removed (if not incorporated) before nucleotides of the same type or different types and labeled with the same single label are introduced.

In some embodiments, a method disclosed herein is a sequencing method, for instance, for DNA or direct RNA sequencing. In some embodiments, the method can use a single detection channel, e.g., for detecting signal intensity of a plurality of different labels. For example, a single channel is sufficient to detect and distinguish signals associated with two fluorophores, ATTO 532 and ATTO 542, based on their characteristic intensity (e.g., sum of relative fluorescence over a range of wavelengths). In some embodiments, the method is a single channel sequencing method. In some embodiments, the method is unterminated and/or non-cyclical. For instance, the method does not require the use of chain terminators (e.g., a reversible terminator that can terminate primer extension reversibly) or sequencing cycles comprising signal deactivation and/or label removal. In some embodiments, the method utilizes labeled nucleotides but the labels do not need to cleaved and/or removed from incorporated nucleotides.

In some embodiments, labeled nucleotides may be added and imaged during incorporation in a real-time sequencing method. A marked spot can be created from the point spread function (PSF) of a single or emitter or group of diffraction limited emitters, for example multiple labels on a single nucleic acid strand. Images may be registered and segmented to identify spot locations. Once a spot is identified, background signal (e.g., due to background fluorescence and/or autofluoresence) may be calculated and removed from images of the spot. Other signal artifacts (for example foreground illumination variation) may be compensated for. A characteristic signal for each spot may be extracted. A number of methods may be used for extracting signals. For example, a characteristic signal may be obtained by extracting the peak value within a spot, and/or by fitting a point spread function (for example a 2D Gaussian function) to the spot profile and using the peak value or other features from the fit. In some embodiments, this characteristic value is termed the “intensity” or “signal intensity” which are used interchangeably herein.

In some embodiments, the intensity of a spot may be extracted over a number of frames to produce an intensity profile (e.g., in the form of a time trace) for a spot. In some embodiments, the intensity profile (e.g., time trace of signal intensity) of a spot is generated from labeled nucleotides incorporated into a strand under synthesis. This profile maybe further corrected and processed to determine a nucleic acid sequence of the complementary template nucleic acid which can be RNA or DNA.

In some embodiments, labeled nucleotides are incorporated into a strand under synthesis (for example using a polymerase or reverse transcriptase). In some embodiments, labeled nucleotides once incorporated do not need to be photobleached before one or more subsequent labeled nucleotides are incorporated. In some examples, first a single nucleic acid strand (5′-AATAG-3′) is attached to a surface and a first labeled nucleotide (“A” in this example) is incorporated using a polymerase or reverse transcriptase. A second labeled nucleotide (“T” in this example) may be present in the sequencing reaction before, during, and/or after the first labeled “A” nucleotide is incorporated. Then, the second labeled “T” nucleotide can be incorporated before the first labeled “A” nucleotide is photobleached. Then, a third labeled nucleotide (“T” in this example) can be incorporated after the first labeled “A” nucleotide is photobleached but before the second labeled “T” nucleotide is photobleached. The third labeled “T” nucleotide can be bleached while the second labeled “T” nucleotide is not yet photobleached. The second labeled “T” nucleotide can then be photobleached. A time trace of the detected signals at the spot can be generated and used to determine a sequence of the nucleic acid strand, e.g., 5′-AAT-3′ which is complementary to the synthesized 5′-ATT-3′ sequence in the sequencing primer strand.

In some embodiments, labeled nucleotides may be incorporated an imaged under illumination (for example objective or prism style TIRF illumination). In some embodiments, labeled nucleotides may be incorporated and photobleaching of the incorporated labeled nucleotides occur stochastically. In some embodiments, nucleotides comprising different bases may be labeled with the same label. In some embodiments, nucleotides comprising different bases may be labeled using labels having different excitation wavelengths and/or different emission wavelengths. In some embodiments, nucleotides comprising different bases may be labeled using labels which result in differing intensity at a given wavelength or across a given range of wavelengths.

In some embodiments, photobleaching and/or any suitable method of dye deactivation may be used. For example, a photocleavable fluorescent nucleotide may be used, for instance, as described in Meng et al., “Design and Synthesis of a Photocleavable Fluorescent Nucleotide 3′-O-Allyl-dGTP-PC-Bodipy-FL-510 as a Reversible Terminator for DNA Sequencing by Synthesis,” J. Org. Chem. 71, 8, 3248-3252 (2006), incorporated herein by reference in its entirety for all purposes. Other methods of dye deactivation based on temperature or pH may also be used.

Photobleachable nucleotides may include 5-(3-Aminoallyl)-2′-deoxyuridine-5′-triphosphate, labeled with ATTO 532, Triethylammonium salt (Jena Biosciences, Germany) or similar ATTO labeled nucleotides. Nucleotides may be introduced at a concentration appropriate to the experimental conditions, for example, 10 nM, 20 nM, 30 nM, 40 nM, 50 nM, 60 nM, 70 nM, 80 nM, 90 nM, or 100 nM, or in a range between any of the aforementioned values. Nucleotides may be constructed where photodamage is used to cause dye cleavage. Nucleotides may also be constructed to contain multiple emitters, providing differing emission strength. Such nucleotides may contain a cleavable element such that all emitters can be simultaneously removed/deactivated.

Nucleotides may be incorporated using a suitable polymerase, for example a 9°N or related polymerase, or Klenow fragment, or the SuperScript® III reverse transcriptase (Invitrogen) or another reverse transcriptase.

In some embodiments, nucleotides are labelled with labels which result in differing intensity. A trace may be extracted from acquired images where nucleotide incorporation and imaging has proceeded as described above using said labels of differing intensity. Such labels result in a convolved signal which photobleaching events occur stochastically. Both incorporation events (increases in intensity) and bleaching events (decreases in intensity) provide information which can aid in determining the nucleotide sequence of a strand under synthesis and the complementary strand being sequenced. Nucleotide labels may be selected such that labels show differing emission levels over the same range of wavelengths. For example ATTO 532 and ATTO 542 may be used which at 537 nm show relative emission levels of 0.443 and 0.104, respectively.

In some embodiments, a method disclosed herein comprises controlling the photobleaching rate, such as by using a free-radical scavenger, for example β-mercaptoethanol (Yanagida et al., 1986, in Applications of Fluorescence in the Biomedical Sciences, Taylor et al. (eds) Adaln R. Liss Inc., New York, pp. 321) or glucose oxidase. For example, in some embodiments, the method comprises tuning the photobleaching rate to keep total emission under a threshold total value. In some embodiments, a method disclosed herein comprises preventing emissions saturating the image sensor well depth at a given exposure time.

A time trace of signal intensity may be analyzed and deconvoluted, for example using a Hidden Markov Model (HMM) capable of decoding a di-nucleotide sequence where nucleotides are labeled with varying brightness. The “A” nucleotide can be labelled with an intensity of magnitude 1 and the “T” nucleotide can be labelled with an intensity of magnitude 2 (double the intensity of “A”). Such an HMM using a Viterbi or other decoder can be used to basecall an intensity trace. The transitions in such a model represent the nucleotide type that is incorporated. The states represent intensity levels obtained from an intensity trace as described above. The transitions labeled Pb represent photobleaching events. The HMM can be used to model any combination of 3 nucleotide types illuminated at any one time. To simplify the example, only 2 nucleotide types are shown here (“A” and “T”), however the model may be extended to 4 nucleotides where more than 3 nucleotide types are illuminated at any one time using known methods. Self-transitions are not shown, which would model a steady state. Additional states may be added to compensate for multiple bleaching events in a single sample. In some embodiments, states may be added to model dye self-quenching, blinking, photo-switching, and/or dye recovery. States may model emission intensity as a fixed value, a range, or as a Gaussian distribution. The transition probabilities for incorporations may be fixed (as determined experimentally) or fitted to each experiment. Similarly, the photobleach transition probabilities (P_{b}) may be fixed (as determined experimentally) or fitted to each experimental dataset.

While the HMM can be demonstrated using two transition types representing adenine (A), thymine (T), it may also be extended with cytosine (C) and guanine (G) nucleotides. The HMM may also represent the sequencing-by-synthesis and photobleaching of a RNA strand.

In some embodiments, a method disclosed herein can be used to provide rapid and inexpensive sequencing solutions, for instance, in response to a pandemic such as COVID-19. Such pandemic scale sequencing methods can rival qPCR based methods in terms of cost, at a cost per run much lower than existing sequencing-by-synthesis methods that rely on flow cell cycles. In some embodiments, the sequencing methods disclosed herein can be used to diagnose a disease or condition, such as viral infection. In some embodiments, the sequencing methods disclosed herein overcome limitations of qPCR based methods and achieve improved detection accuracy.

In some embodiments, provided herein are low-cost sequencing methods (e.g., for pandemic response) that can accurately detect a plurality of nucleic acid molecules, including viral RNA in a biological sample. For instance, the biological sample can be processed to extract viral nucleic acid (e.g., RNA) while optionally depleting human nucleic acid (e.g., RNA). The extracted viral nucleic acid can be sequenced using a method disclosed herein in a massively parallel, high throughput manner. As such, the present/absence, amount, and sequence of viral nucleic acid can be rapidly detected using a method comprising RNA extraction from patient samples and direct RNA sequencing according to some embodiments of the present disclosure. In some embodiments, no reverse transcription of RNA to cDNA is required. In some embodiments, no multiplex PCR of the extracted RNA or cDNA reverse transcribed therefrom is required. In some embodiments, no further processing of the extracted nucleic acid (e.g., RNA) is required prior to sequencing using a method disclosed herein. For instance, in some embodiments, the extracted nucleic acid (e.g., RNA) does not need to be tagmented and/or amplified prior to sequencing. In some embodiments, a method provided herein can be used to sequence at least about 10, 11, 12, 13, 14, 15, 16, 17, 18, 19, 20, 21, 22, 23, 24, 25, 26, 27, 28, 29, or 30 consecutive nucleotides or longer nucleotide sequences, with less than about 10%, less than about 5%, or less than about 1% error rate in between about 100,000 and about 1 million sequencing reads.

### II. Samples and Nucleic Acid Molecules

The nucleic acid molecules used in the methods described herein may be obtained from any suitable biological source, for example a tissue sample, a blood sample, a plasma sample, a saliva sample, a fecal sample, or a urine sample. The polynucleotides may be DNA or RNA molecules. In some embodiments, RNA molecules are reverse transcribed into DNA molecules prior to hybridizing the polynucleotide to a sequencing primer. In some embodiments, RNA molecules are not reverse transcribed and are hybridized to a sequencing primer for direct RNA sequencing. In some embodiments, the nucleic acid molecule is a cell-free DNA (cfDNA), such as a circulating tumor DNA (ctDNA) or a fetal cell-free DNA.

Examples of nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid molecules also include RNA molecules such as various types of coding and non-coding RNA, including viral RNAs. Examples of the different types of RNA molecules include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the nucleic acid molecules disclosed herein are non-capped mRNA, a non-polyadenylated mRNA, and a non-spliced mRNA. The RNA analyte can be a transcript of another nucleic acid molecule (e.g., DNA or RNA such as viral RNA).

In some embodiments, a nucleic acid molecule may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In some embodiments, a nucleic acid molecule can be extracted from a cell, a virus, or a tissue sample comprising the cell or virus. Processing conditions can be adjusted to extract or release nucleic acid molecules (e.g., RNA) from a cell, a virus, or a tissue sample.

### III. Sequencing Methods

In some embodiments, disclosed herein is a method for nucleic acid sequencing comprising colony surface amplification (e.g., using bridge amplification or an isothermal amplification method). Exemplary colony surface amplification methods include those disclosed in U.S. Pat. No. 7,115,400, U.S. Pat. No. 7,541,444, U.S. Pat. No. 7,771,973, U.S. Pat. No. 8,071,739, U.S. Pat. No. 8,597,881, U.S. Pat. No. 8,652,810, U.S. Pat. No. 9,121,060, U.S. Pat. No. 9,297,006, U.S. Pat. No. 9,388,464, U.S. Pat. No. 10,370,652, U.S. Pat. No. 10,513,731, and US 2020/0399692, each incorporated herein by reference in its entirety for all purposes.

In some embodiments, an amplified cluster of nucleic acid molecules (e.g., DNA) is created on a surface. In some embodiments, an amplified cluster is clonal and all nucleic acid strands in the cluster comprise at least one identical sequence to be determined, accepting polymerase errors (e.g., if a nucleotide difference is introduced due to polymerase error during clonal amplification, the sequences in two strand can be considered an identical sequence). In some embodiments, an amplified cluster can comprise sequences from one or more concatemers, such as a rolling circle amplification product comprising multiple copies or repeats of a unit sequence, and the copies or repeats comprise at least one identical sequence to be determined and can be cleaved from the rolling circle amplification product.

In some embodiments, a cluster and an identical sequence shared among molecules (or shared by repeats in the same molecule) can be sequenced, e.g., using sequencing-by-synthesis (SBS), sequencing-by-binding (SBB) or sequencing using a dye labeled polymer with multiple, identical nucleotides attached (e.g., avidity sequencing).

In some embodiments, in a method disclosed herein, reversibly terminated nucleotides are incorporated into a strand under synthesis using a polymerase. In some embodiments, the nucleotides are labeled with a cleavable fluorophore, such that each nucleotide type may be specifically detected. Once detected, the label may be removed, and the blocking group (e.g., a terminator) can be removed. In some embodiments, subsequent nucleotides may be incorporated and the complete sequence of the identical sequences in the strands in the cluster is determined.

In some embodiments, provided herein is a cluster based amplification approach. Cluster based sequencing generally provides more emitted signals than available with conventional single molecule approaches. Cluster based amplification can provide advantages in terms of improved signal-to-noise (SNR) ratios and allows cheaper and simpler cameras to be used. The approach also means that a certain amount of photo-damage may be tolerated. If a fraction of molecules (strands) within a cluster are photodamaged, the remaining molecules may still provide sufficient signal to allow sequencing to continue and the sequence to be determined. However, a major limitation of cluster based sequencing is phasing.

In certain sequencing-by-synthesis methods, a first population of detectably labeled nucleotides (e.g., dNTP) are introduced into a reaction chamber to contact a template nucleotide hybridized to a sequencing primer in the chamber, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced into the reaction chamber (e.g., a flow cell), but in order for a second detectably labeled nucleotide to incorporate into the extended sequencing primer, nucleotides in the first population of nucleotides that have not incorporated into a sequencing primer generally must be removed (e.g., by washing), and a second population of detectably labeled nucleotides must be introduced into the chamber. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in these methods, cycles of introducing and removing detectably labeled nucleotides must be performed.

In contrast, in one embodiment of a method disclosed herein, the first detectably labeled nucleotide and the second detectably labeled nucleotide do not need to be introduced into the chamber in separate cycles. In some embodiments, the second detectably labeled nucleotide is already present in the reaction chamber when the first detectably labeled nucleotide is being incorporated into the sequencing primer. In some embodiments, other molecules of the first detectably labeled nucleotide that have not incorporated into a template nucleotide/sequencing primer duplex immobilized at a particular location are not removed when the second detectably labeled nucleotide is incorporated into the extended sequencing primer. In fact, the second detectably labeled nucleotide can be a molecule of the first detectably labeled nucleotide that has not incorporated. For instance, the first detectably labeled nucleotide can be an A nucleotide, and another A nucleotide can be the second detectably labeled nucleotide. Thus, for a template nucleotide/sequencing primer duplex at a given location, it can be said that detectably labeled nucleotides are continuously incorporated in a noncyclic manner.

In any of the embodiments herein, the template nucleotide for a sequencing method disclosed herein can be in a decimated cluster, where some template nucleotides in the same cluster have been deactivated such that the deactivated strands do not give rise to signals associated with nucleotide incorporation or nonincorporated events and the deactivated strands remain “dark” throughout the single nucleotide, real-time sequencing of strands within the cluster that are not deactivated.

In some embodiments, provided herein is a method of obtaining the forward and reverse strand sequence of a double-stranded nucleic acid, such as a dsDNA. In some embodiments, the double-stranded nucleic acid (e.g., dsDNA) is weakly confined on a surface, such as a surface of a solid substrate, e.g., a flowcell. In some embodiments, the method comprises providing a single-stranded nucleic acid from the double-stranded nucleic acid, and the provided single-stranded nucleic acid (e.g., ssDNA) is attached to the surface. In some embodiments, one or both of the single-stranded nucleic acids are within a distance (e.g., a fixed distance of no more than about 8 μm, no more than about 6 μm, no more than about 4 μm, no more than about 2 μm, no more than about 1 μm, no more than about 0.5 μm, or no more than about 0.25 μm) from the location on the surface of the original source double-stranded nucleic acid. In some embodiments, the single-stranded nucleic acid is immobilized or fixed at a location on the surface.

In some embodiments, the single-stranded nucleic acid (e.g., a ssDNA immobilized or fixed at a location on the surface) is further amplified, e.g., into clusters or a concatemer (e.g., a long continuous DNA molecule that contains multiple copies of the same DNA sequence linked in series), for example using a bridge amplification process or rolling circle amplification. In some embodiments, the single-stranded nucleic acid and amplicons thereof are sequenced to obtain spatially localized reads of the forward and reverse strand resulting from the same double-stranded nucleic acid (e.g., dsDNA), e.g., a dsDNA fragment of a genome. In some embodiments, the sequences of the forward and reverse strands are used (e.g., the sequencing reads and consensus sequences generated from sequencing reads can be compared and/or combined) to generate a single higher accuracy read. In some embodiments, the sequences of the forward and reverse strands are combined to generate a single longer read.

In some embodiments, a model-based algorithm is used to combine signals from the forward and reverse strands to create a single higher accuracy read. In some embodiments, other features extract from the image (for example circularity) are used in the model based basecalling process.

In some embodiments, a double-stranded nucleic acid (e.g., dsDNA) is attracted to the surface using an electric field. In some embodiments, a single-stranded nucleic acid (e.g., ssDNA) is attracted to the surface using an electric field.

In some embodiments, a single-stranded nucleic acid (e.g., ssDNA) is confined using a charge/field barrier. In some embodiments, a double-stranded nucleic acid (e.g., dsDNA) is attracted to the surface, a single-stranded nucleic acid (e.g., ssDNA) is generated from the double-stranded nucleic acid, and the single-stranded nucleic acid is confined at a location on the surface, e.g., using a charge/field barrier.

In some embodiments, signals from the forward and reverse single-stranded nucleic acids (e.g., from the same double-stranded nucleic acid) cannot be resolved, and a mix signal is generated and detected at a location on the surface. In some embodiments, the mixed signal is base called using a model-based algorithm.

In some embodiments, provided herein is a method where cells are attached (e.g., directly or indirectly, covalently or noncovelently) to a surface (e.g., using a method disclosed herein or any other suitable known method), where the cell is broken (e.g., the cell's membrane and/or nuclear envelope is disrupted, e.g., by an agent or a condition that disrupted lipid bilayers), and cellular material is confined at a location on the surface using electric fields.

In some embodiments, a negative field around the cellular attachment site is used to confine cellular material spatially on the surface. In some embodiments, the cellular material is attracted to the surface. In some embodiments, a positive field is used to attract cellular material (e.g., nucleic acid such as DNA and/or RNA which is negatively charged) to a location on the surface, and reduce diffusion of the cellular material away from the location on the surface. In some embodiments, indexes are introduced into cellular DNA/RNA once confined.

### A. Nucleotides and Nucleotide Analogs

In some embodiments, a method disclosed herein comprises using one or more nucleotides or analogs thereof, including a native nucleotide or a nucleotide analog or modified nucleotide (e.g., labeled with one or more detectable labels). In some embodiments, a nucleotide analog comprises a nitrogenous base, five-carbon sugar, and phosphate group, wherein any component of the nucleotide may be modified and/or replaced. In some embodiments, a method disclosed herein may comprise but does not require using one or more non-incorporable nucleotides. Non-incorporable nucleotides may be modified to become incorporable at any point during the sequencing method.

Nucleotide analogs include, but are not limited to, alpha-phosphate modified nucleotides, alpha-beta nucleotide analogs, beta-phosphate modified nucleotides, beta-gamma nucleotide analogs, gamma-phosphate modified nucleotides, caged nucleotides, or ddNTPs. Examples of nucleotide analogs are described in U.S. Pat. No. 8,071,755, which is incorporated by reference herein in its entirety.

In some embodiments, a method disclosed herein may comprise but does not require using terminators that reversibly prevent nucleotide incorporation at the 3′-end of the primer. One type of reversible terminator is a 3′-O-blocked reversible terminator. Here the terminator moiety is linked to the oxygen atom of the 3′-OH end of the 5-carbon sugar of a nucleotide. For example, U.S. Pat. Nos. 7,544,794 and 8,034,923 (the disclosures of these patents are incorporated by reference) describe reversible terminator dNTPs having the 3′-OH group replaced by a 3′-ONH₂ group. Another type of reversible terminator is a 3′-unblocked reversible terminator, wherein the terminator moiety is linked to the nitrogenous base of a nucleotide. For example, U.S. Pat. No. 8,808,989 (the disclosure of which is incorporated by reference) discloses particular examples of base-modified reversible terminator nucleotides that may be used in connection with the methods described herein. Other reversible terminators that similarly can be used in connection with the methods described herein include those described in U.S. Pat. Nos. 7,956,171, 8,071,755, and 9,399,798, herein incorporated by reference.

In some embodiments, a method disclosed herein may comprise but does not require using nucleotide analogs having terminator moieties that irreversibly prevent nucleotide incorporation at the 3′-end of the primer. Irreversible nucleotide analogs include 2′, 3′-dideoxynucleotides, ddNTPs (ddGTP, ddATP, ddTTP, ddCTP). Dideoxynucleotides lack the 3′-OH group of dNTPs that is essential for polymerase-mediated synthesis.

In some embodiments, a method disclosed herein may comprise but does not require using non-incorporable nucleotides comprising a blocking moiety that inhibits or prevents the nucleotide from forming a covalent linkage to a second nucleotide (3′-OH of a primer) during the incorporation step of a nucleic acid polymerization reaction. The blocking moiety can be removed from the nucleotide, allowing for nucleotide incorporation.

In some embodiments, a method disclosed herein may comprise but does not require using 1, 2, 3, 4 or more nucleotide analogs present in the SBS reaction. In some embodiments, a nucleotide analog is replaced, diluted, or sequestered during an incorporation step. In some embodiments, a nucleotide analog is replaced with a native nucleotide. In some embodiments, a nucleotide analog is modified during an incorporation step. The modified nucleotide analog can be similar to or the same as a native nucleotide.

In some embodiments, a method disclosed herein may comprise but does not require using a nucleotide analog having a different binding affinity for a polymerase than a native nucleotide. In some embodiments, a nucleotide analog has a different interaction with a next base than a native nucleotide. Nucleotide analogs and/or non-incorporable nucleotides may base-pair with a complementary base of a template nucleic acid.

In some embodiments, one or more nucleotides can be labeled with distinguishing and/or detectable tags or labels. The tags may be distinguishable by means of their differences in fluorescence, Raman spectrum, charge, mass, refractive index, luminescence, length, or any other measurable property. The tag may be attached to one or more different positions on the nucleotide, so long as the fidelity of binding to the polymerase-nucleic acid complex is sufficiently maintained to enable identification of the complementary base on the template nucleic acid correctly. In some embodiments, the tag is attached to the nucleobase of the nucleotide. Alternatively, a tag is attached to the gamma phosphate position of the nucleotide.

Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to another moiety, for example, a nucleotide or nucleotide analog, and can include a fluorescent, a colorimetric, or a chemiluminescent label.

In some embodiments, a detectable label can be attached to another moiety, for example, a nucleotide or nucleotide analog. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X. BODIPY® 530/550, BODIPY® 558/568, BODIPY® 564/570, BODIPY® 581/591. BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GencBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DIA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP. EGFP. ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GencBLAzer™ (CCF2), GFP Red Shifted (rsGFP), GFP Wild Type, GFP/BFP FRET, GFP/DsRed FRET. Hoechst 33342 & 33258, 7-Hydroxy-4-methylcoumarin (pH 9), 1.5 IAEDANS, Indo-1 (high calcium), Indo-1 (low calcium), Indodicarbocyanine, Indotricarbocyanine, JC-1, 6-JOE, JOJO™-1/JO-PROTM-1. LDS 751 (+DNA), LDS 751 (+RNA), LOLO™-1/LO-PRO™-1. Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Bluc, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard. R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1. SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X. Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 542, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

The detectable label can be directly detectable by itself (e.g., radioisotope labels or fluorescent labels) or, in the case of an enzymatic label, can be indirectly detectable, c.g., by catalyzing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

### B. Polymerases

Polymerases that may be used to carry out the disclosed techniques include naturally-occurring polymerases and any modified variations thereof, including, but not limited to, mutants, recombinants, fusions, genetic modifications, chemical modifications, synthetics, and analogs. Naturally occurring polymerases and modified variations thereof are not limited to polymerases that retain the ability to catalyze a polymerization reaction. In some embodiments, the naturally occurring and/or modified variations thereof retain the ability to catalyze a polymerization reaction. In some embodiments, the naturally-occurring and/or modified variations have special properties that enhance their ability to sequence DNA, including enhanced binding affinity to nucleic acids, reduced binding affinity to nucleic acids, enhanced catalysis rates, reduced catalysis rates, etc. Mutant polymerases include polymerases wherein one or more amino acids are replaced with other amino acids (naturally or non-naturally occurring), and insertions or deletions of one or more amino acids.

In some embodiments, a method disclosed herein may comprise but does not require using modified polymerases containing an external tag (e.g., an exogenous detectable label), which can be used to monitor the presence and interactions of the polymerase. In some embodiments, intrinsic signals from the polymerase can be used to monitor their presence and interactions. Thus, the provided methods can include monitoring the interaction of the polymerase, nucleotide and template nucleic acid through detection of an intrinsic signal from the polymerase. In some embodiments, the intrinsic signal is a light scattering signal. For example, intrinsic signals include native fluorescence of certain amino acids such as tryptophan.

In some embodiments, a method disclosed herein may comprise using an unlabeled polymerase, and monitoring is performed in the absence of an exogenous detectable label associated with the polymerase. Some modified polymerases or naturally occurring polymerases, under specific reaction conditions, may incorporate only single nucleotides and may remain bound to the primer-template after the incorporation of the single nucleotide.

In some embodiments, a method disclosed herein may comprise using an polymerase unlabeled with an exogenous detectable label (e.g., a fluorescent label). The label can be chemically linked to the structure of the polymerase by a covalent bond after the polymerase has been at least partially purified using protein isolation techniques. For example, the exogenous detectable label can be chemically linked to the polymerase using a free sulfhydryl or a free amine moiety of the polymerase. This can involve chemical linkage to the polymerase through the side chain of a cysteine residue, or through the free amino group of the N-terminus. In certain preferred embodiments, a fluorescent label attached to the polymerase is useful for locating the polymerase, as may be important for determining whether or not the polymerase has localized to a spot on an array corresponding to immobilized primed template nucleic acid. The fluorescent signal need not, and in some embodiments does not change absorption or emission characteristics as the result of binding any nucleotide. In some embodiments, the signal emitted by the labeled polymerase is maintained uniformly in the presence and absence of any nucleotide being investigated as a possible next correct nucleotide.

The term polymerase and its variants, as used herein, also refers to fusion proteins comprising at least two portions linked to each other, for example, where one portion comprises a peptide that can catalyze the polymerization of nucleotides into a nucleic acid strand is linked to another portion that comprises a second moiety, such as, a reporter enzyme or a processivity-modifying domain. For example, T7 DNA polymerase comprises a nucleic acid polymerizing domain and a thioredoxin binding domain, wherein thioredoxin binding enhances the processivity of the polymerase. Absent the thioredoxin binding, T7 DNA polymerase is a distributive polymerase with processivity of only one to a few bases. Although DNA polymerases differ in detail, they have a similar overall shape of a hand with specific regions referred to as the fingers, the palm, and the thumb; and a similar overall structural transition, comprising the movement of the thumb and/or finger domains, during the synthesis of nucleic acids.

DNA polymerases include, but are not limited to, bacterial DNA polymerases, eukaryotic DNA polymerases, archacal DNA polymerases, viral DNA polymerases and phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, ϵ, η, ζ, λ, σ, μ, and κ, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cp7 DNA polymerase, T7 DNA polymerase, and T4 polymerase. Other DNA polymerases include thermostable and/or thermophilic DNA polymerases such as DNA polymerases isolated from Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. Engineered and modified polymerases also are useful in connection with the disclosed techniques. For example, modified versions of the extremely thermophilic marine archaca Thermococcus species 9° N (e.g., Therminator DNA polymerase from New England BioLabs Inc.; Ipswich, Mass.) can be used. Still other useful DNA polymerases, including the 3PDX polymerase are disclosed in U.S. Pat. No. 8,703,461, the disclosure of which is incorporated by reference in its entirety.

RNA polymerases include, but are not limited to, viral RNA polymerases such as T7 RNA polymerase, T3 polymerase, SP6 polymerase, and K11 polymerase; Eukaryotic RNA polymerases such as RNA polymerase I, RNA polymerase II, RNA polymerase III, RNA polymerase IV, and RNA polymerase V; and Archaea RNA polymerase.

Reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase that maintains the telomeres of cukaryotic chromosomes.

### C. Sequencing Reactions

In some embodiments of a sequencing-by-synthesis (SBS) method disclosed herein, a first labeled nucleotide that has been incorporated is not deactivated (e.g., by removal and/or photobleaching of the label) prior to the introduction and/or incorporation of the next, second labeled nucleotide. The first and second labeled nucleotides can comprise the same base or different bases. The first and second labeled nucleotides can be introduced into a sequencing reaction mix simultaneously or at different time points in any order. Further, the first and second labeled nucleotides can be introduced by itself (e.g., in a suitable solvent such as water) or in a mixture with another sequencing reagent, such as one or more other labeled nucleotides and/or one or more unlabeled nucleotides. The first and second labeled nucleotides can also comprise the same base or different bases. In some embodiments, nucleotides that have not been incorporated at a residue corresponding to a base in the template nucleic acid (e.g., because the first labeled nucleotide has been incorporated at that residue) are not removed from the sequencing reaction mix prior to the introduction and/or incorporation of the second labeled nucleotide. In some embodiments, the first and second labeled nucleotides (and optionally labeled nucleotides for interrogating subsequent bases in the template) are provided in the same sequencing reaction mix, and the first, second, and optionally any subsequent labeled nucleotide(s) are incorporated sequentially in a continuous manner.

Thus, unlike existing SBS methods, some embodiments of the method disclosed herein use continuous introduction and/or incorporation of nucleotides (e.g., fluorescently labeled A, T, C, and/or G nucleotides) without the need of label deactivation and/or wash steps in between sequential incorporation events for a given template nucleic acid molecule to be sequenced. Rather, in some embodiments, label deactivation (e.g., by cleaving and/or photobleaching the label) of a first incorporated nucleotide may occur stochastically throughout the continuous nucleotide incorporation process, for instance, prior to, during, or after the incorporation of a second, third, fourth, or a subsequent labeled nucleotide.

Nucleic acid sequencing reaction mixtures, or simply “reaction mixtures,” typically include reagents that are commonly present in polymerase based nucleic acid synthesis reactions. The reaction mixture can include other molecules including, but not limited to, enzymes. In some embodiments, the reaction mixture comprises any reagents or biomolecules generally present in a nucleic acid polymerization reaction. Reaction components may include, but are not limited to, salts, buffers, small molecules, detergents, crowding agents, metals, and ions. In some embodiments, properties of the reaction mixture may be manipulated, for example, electrically, magnetically, and/or with vibration.

The provided methods herein may further comprise but do not require one or more wash steps; a temperature change; a mechanical vibration; a pH change; or an optical stimulation that is not dye illumination or photobleaching. In some embodiments, the wash step comprises contacting the substrate and the nucleic acid molecule, the primer, and/or the polymerase with one of more buffers, detergents, protein denaturants, proteases, oxidizing agents, reducing agents, or other agents capable of crosslinking or releasing crosslinks, c.g., crosslinks within a polymerase or crosslinks between a polymerase and nucleic acid. Methods and compositions for nucleic acid sequencing are known, for example, as described in U.S. Pat. Nos. 10,246,744 and 10,844,428, incorporated herein by reference in their entireties for all purposes.

Reaction mixture reagents can include, but are not limited to, enzymes (e.g., polymerase), dNTPs, template nucleic acids, primer nucleic acids, salts, buffers, small molecules, co-factors, metals, and ions. The ions may be catalytic ions, divalent catalytic ions, non-catalytic ions, non-covalent metal ions, or a combination thereof. The reaction mixture can include salts, such as NaCl, KCl, potassium acetate, ammonium acetate, potassium glutamate, or NH₄Cl or the like, that ionize in aqueous solution to yield monovalent cations. The reaction mixture can include a source of ions, such as Mg²⁺, Mn²⁺, Co²⁺, Cd²⁺, and/or Ba²⁺ ions. The reaction mixture can include tin, Ca²⁺, Zn²⁺, Cu²⁺, Co²⁺, Fe²⁺, and/or Ni²⁺, or other divalent non-catalytic metal cations. In some embodiments, the reaction mixture can include metal cations that may inhibit formation of phosphodiester bonds between the primed template nucleic acid molecule and the cognate nucleotide. In some embodiments, the metal cations can be used (e.g., at a suitable concentration) to slow down but not completely inhibit or prevent nucleotide incorporation, thereby reducing multiple nucleotide incorporation events in a single detection window.

In some embodiments, the sequencing reaction conditions comprise contacting the nucleic acid molecule and the primer with a buffer that regulates osmotic pressure. In some embodiments, the reaction mixture comprises a buffer that regulates osmotic pressure. In some embodiments, the buffer is a high salt buffer that includes a monovalent ion, such as a monovalent metal ion (e.g., potassium ion or sodium ion) at a concentration of from about 50 to about 1,500 mM. Salt concentrations in the range of from about 100 to about 1,500 mM, or from about 200 to 1,000 mM may also be used. In some embodiments, the buffer further comprises a source of glutamate ions (e.g., potassium glutamate). In some embodiments, the buffer comprises a stabilizing agent. In some embodiments, the stabilizing agent is a non-catalytic metal ion (e.g., a divalent non-catalytic metal ion). Non-catalytic metal ions useful in this context include, but are not limited to, calcium, strontium, scandium, titanium, vanadium, chromium, iron, cobalt, nickel, copper, zinc, gallium, germanium, arsenic, selenium, rhodium, europium, and/or terbium. In some embodiments, the non-catalytic metal ion is strontium, tin, or nickel. In some embodiments, the sequencing reaction mixture comprises strontium chloride or nickel chloride. In some embodiments, the stabilizing agent can be used (e.g., at a suitable concentration) to slow down but not completely inhibit or prevent nucleotide incorporation, thereby reducing multiple nucleotide incorporation events in a single detection window.

The buffer can include Tris, Tricine, HEPES, MOPS, ACES, MES, phosphate-based buffers, and acetate-based buffers. The reaction mixture can include chelating agents such as EDTA, EGTA, and the like. In some embodiments, the reaction mixture includes cross-linking reagents.

In some embodiments, the interaction between the polymerase and template nucleic acid may be manipulated by modulating sequencing reaction parameters such as ionic strength, pH, temperature, or any combination thereof, or by the addition of a destabilizing agent to the reaction. In some embodiments, the destabilizing agent can be used (e.g., at a suitable concentration) to slow down but not completely inhibit or prevent nucleotide incorporation, thereby reducing multiple nucleotide incorporation events in a single detection window.

In some embodiments, high salt (e.g., 50 to 1,500 mM) and/or pH changes are utilized to destabilize a complex between the polymerase and template nucleic acid. In some embodiments, the reaction conditions favor the stabilization of a complex among the polymerase, the template nucleic acid, and a labeled nucleotide. By way of example, the pH of the reaction mixture can be adjusted from 4.0 to 10.0 to favor the stabilization of a complex among the polymerase, the template nucleic acid, and a labeled nucleotide. In some embodiments, the pH of the reaction mixture is from 4.0 to 6.0. In some embodiments, the pH of the reaction mixture is 6.0 to 10.0. In some embodiments, a suitable salt concentration and/or a suitable pH can be selected to slow down but not completely inhibit or prevent nucleotide incorporation, thereby reducing multiple nucleotide incorporation events in a single detection window.

In some embodiments, the reaction mixture comprises a competitive inhibitor, where the competitive inhibitor may reduce the occurrence of multiple incorporations events in a detection window. In one embodiment, the competitive inhibitor is a non-incorporable nucleotide. In an embodiment, the competitive inhibitor is an aminoglycoside. The competitive inhibitor is capable of replacing either the nucleotide or the catalytic metal ion in the active site, such that the competitive inhibitor occupies the active site preventing or slowing down a nucleotide incorporation. In some embodiments, both an incorporable nucleotide and a competitive inhibitor are introduced, such that the ratio of the incorporable nucleotide and the inhibitor can be adjusted to modulate the rate of incorporation of a single nucleotide at the 3′-end of the primer. In some embodiments, the competitive inhibitor can be used (e.g., at a low concentration) to slow down but not completely inhibit or prevent nucleotide incorporation, thereby reducing multiple nucleotide incorporation events in a single detection window.

In some embodiments, the reaction mixture comprises at least one nucleotide molecule that is a non-incorporable nucleotide. In some embodiments, the reaction mixture comprises one or more nucleotide molecules incapable of incorporation into the primer of the primed template nucleic acid molecule. Such nucleotides incapable of incorporation include, for example, monophosphate nucleotides. For example, the nucleotide may contain modifications to the triphosphate group that make the nucleotide non-incorporable. Examples of non-incorporable nucleotides may be found in U.S. Pat. No. 7,482,120, which is incorporated by reference herein in its entirety. In some embodiments, the primer may not contain a free hydroxyl group at its 3′-end, thereby rendering the primer incapable of incorporating any nucleotide, and, thus, making any nucleotide non-incorporable. In some embodiments, the primer may be processed such that it contains a free hydroxyl group at its 3′-end to allow nucleotide incorporation. In some embodiments, the non-incorporable nucleotide can be used (e.g., at a low concentration) to slow down but not completely inhibit or prevent nucleotide incorporation, thereby reducing multiple nucleotide incorporation events in a single detection window.

In some embodiments, the reaction mixture comprises at least one nucleotide molecule that is incorporable but is incorporated at a slower rate compared to a corresponding naturally-occurring nucleoside triphosphate (e.g., NTP or dNTP). Such nucleotides incorporable at a slower rate may include, for example, diphosphate nucleotides. For example, the nucleotide may contain modifications to the triphosphate group that make the nucleotide incorporable at a slower rate. In some embodiments, the nucleotide incorporable at a slower rate can be used to slow down but not completely inhibit or prevent nucleotide incorporation, thereby reducing multiple nucleotide incorporation events in a single detection window.

In some embodiments, the reaction mixture comprises a polymerase inhibitor. In some embodiments, the polymerase inhibitor is a pyrophosphate analog. In some embodiments, the polymerase inhibitor is an allosteric inhibitor. In some embodiments, the polymerase inhibitor is a DNA or an RNA aptamer. In some embodiments, the polymerase inhibitor competes with a catalytic-ion binding site in the polymerase. In some embodiments, the polymerase inhibitor is a reverse transcriptase inhibitor. The polymerase inhibitor may be an HIV-1 reverse transcriptase inhibitor or an HIV-2 reverse transcriptase inhibitor. The HIV-1 reverse transcriptase inhibitor may be a (4/6-halogen/MeO/EtO-substituted benzo [d]thiazol-2-yl) thiazolidin-4-one. In some embodiments, the polymerase inhibitor can be used (e.g., at a low concentration) to slow down but not completely inhibit or prevent nucleotide incorporation, thereby reducing multiple nucleotide incorporation events in a single detection window.

In some embodiments, the contacting step is facilitated by the use of a chamber such as a flow cell. The methods and apparatus described herein may employ next generation sequencing technology (NGS), which allows massively parallel sequencing. In some embodiments, single DNA molecules are sequenced in a massively parallel fashion within a reaction chamber. A flow cell may be used but is not necessary. Flowing liquid reagents through the flow cell, which contains an interior solid support surface (e.g., a planar surface), conveniently permits reagent exchange. Immobilized to the interior surface of the flow cell is one or more primed template nucleic acids to be sequenced or interrogated using the procedures described herein. Typical flow cells include microfluidic valving that permits delivery of liquid reagents (e.g., components of the “reaction mixtures” discussed herein) to an entry port. Liquid reagents can be removed from the flow cell by exiting through an exit port.

In some embodiments, a reaction chamber disclosed herein can comprise a reagent wall, an imaging area, and optionally an outlet configured to remove molecules of one or more of the polymerase, the first detectably labeled nucleotide, the second detectably labeled nucleotide, and/or one or more other reagents from the imaging area. In some embodiments, the device may comprise one or more vents but no outlet or exit port for the reaction mixture. In some embodiments, a method disclosed herein does not comprise a step of removing liquid reagents through an outlet or exit port, e.g., from a reaction chamber such as a flow cell.

The methods disclosed herein may but do not need to be used in combination with any NGS sequencing methods. The sequencing technologies of NGS include but are not limited to pyrosequencing, sequencing-by-synthesis with reversible dye terminators, sequencing by oligonucleotide probe ligation, and ion semiconductor sequencing. Nucleic acids such as DNA or RNA from individual samples can be sequenced individually (singleplex sequencing) or nucleic acids such as DNA or RNA from multiple samples can be pooled and sequenced as indexed genomic molecules (multiplex sequencing) on a single sequencing run, to generate up to several hundred million reads of sequences. Examples of sequencing technologies that can be used to obtain the sequence information according to the present method are further described here.

Some sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.). In addition to the single molecule sequencing performed using sequencing-by-synthesis of Helicos Biosciences, other single molecule sequencing technologies include, but are not limited to, the SMRTTM technology of Pacific Biosciences, the ION TORRENT™ technology, and nanopore sequencing developed for example, by Oxford Nanopore Technologies.

While the automated Sanger method is considered as a ‘first generation’ technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM).

In some embodiments, the disclosed methods may be used in combination with massively parallel sequencing of nucleic acid molecules using Illumina's sequencing-by-synthesis and reversible terminator-based sequencing chemistry. In some implementation, a method disclosed herein can use a flow cell having a glass slide with lanes.

After sequencing of nucleic acid molecules, sequence reads of predetermined length, c.g., at least about 15 bp, are localized by mapping (alignment) to a known reference sequence or genome (e.g., viral sequences or genomes). A number of computer algorithms are available for aligning sequences, including without limitation BLAST, BLITZ, FASTA, BOWTIE, or ELAND (Illumina, Inc., San Diego, Calif., USA).

In one illustrative, but non-limiting, embodiment, the methods described herein may comprise obtaining sequence information for the nucleic acids in a test sample, for example, using single molecule sequencing technology similar to the Helicos True Single Molecule Sequencing (tSMS) technology. In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. In some embodiments the templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are discerned by imaging the flow cell surface. After imaging, a cleavage step removes the fluorescent label, and the process is repeated with other fluorescently labeled nucleotides until the desired read length is achieved. Sequence information is collected with each nucleotide addition step. Whole genome sequencing by single molecule sequencing technologies excludes or typically obviates PCR-based amplification in the preparation of the sequencing libraries, and the methods allow for direct measurement of the sample, rather than measurement of copies of that sample.

In another illustrative, but non-limiting, embodiment, the methods described herein may comprise obtaining sequence information for the nucleic acids in the test sample, similar to the single molecule, real-time (SMRT™) sequencing technology of Pacific Biosciences. In SMRT sequencing, the continuous incorporation of dye-labeled nucleotides is imaged during DNA synthesis. Single DNA polymerase molecules are attached to the bottom surface of individual zero-mode wavelength detectors (ZMW detectors) that obtain sequence information while phospholinked nucleotides are being incorporated into the growing primer strand. A ZMW detector includes a confinement structure that enables observation of incorporation of a single nucleotide by DNA polymerase against a background of fluorescent nucleotides that rapidly diffuse in an out of the ZMW (e.g., in microseconds). It typically takes several milliseconds to incorporate a nucleotide into a growing strand. During this time, the fluorescent label is excited and produces a fluorescent signal, and the fluorescent tag is cleaved off. Measurement of the corresponding fluorescence of the dye indicates which base was incorporated. The process is repeated to provide a sequence.

In some embodiments, the provided sequencing methods disclosed herein may regulate polymerase interaction with the nucleotides and template nucleic acid (as well as rate of nucleotide incorporation) in a manner that reveals the identity of the next base while controlling the chemical addition of a nucleotide. In some embodiments, the SBS reaction condition comprises a plurality of primed template nucleic acids, polymerases, nucleotides, or any combination thereof. In some embodiments, the plurality of nucleotides comprises 1, 2, 3, 4, or more types of different nucleotides, for example dATP, dTTP (or dUTP), dGTP, and dCTP. In some embodiments, the plurality of template nucleic acids are single molecules immobilized on a substrate for single molecule sequencing.

In some embodiments, the method can further comprise contacting the nucleic acid molecule with the substrate to immobilize the nucleic acid molecule. In some embodiments, the nucleic acid molecule can be immobilized at a density of one molecule per at least about 250 nm², at least about 200 nm², at least about 150 nm², at least about 100 nm², at least about 90 nm², at least about 80 nm², at least about 70 nm², at least about 60 nm², at least about 50 nm², at least about 40 nm², at least about 30 nm², at least about 20 nm², at least about 10 nm², at least about 5 nm², or in between any two of the aforementioned values. Methods and compositions for arraying biomolecules on a substrate, e.g., as described in US 2005/0042649 (incorporated herein by reference in its entirety for all purposes), may be used in methods disclosed herein.

In some embodiments, nucleic acid molecules, polymerase molecules, and/or sequencing primers can be provided on the substrate for super-resolution signal detection. For instance, two nucleic acid molecules to be sequenced may be at two spots near each other. If only one spot is emitting at any one time, a localization based technique may be used to resolve the spot locations to sub-diffraction limited resolution, thereby assigning detected signals (e.g., emissions) to different molecules/strands under synthesis. In such cases, nucleic acid molecules to be sequenced may be packed on the substrate at a density of about one molecule per 20 nm², one molecule per 15 nm², one molecule per 10 nm², at least about 5 nm², or even higher density.

In some embodiments, the detectable labels may comprise one or more labels that blink which may be used to achieve super-resolution localization of nucleic acid strands being sequenced during sequencing at the single molecule level. In some embodiments, labels with differing blinking characteristics may be used for labeling one or more nucleotides. In some embodiments, the detectable labels may comprise one or more labels that exhibit stochastic blinking (also known as photoluminescence intermittence), such as quantum dots. The phenomenon of blinking may be due to high excitation power resulting in a local electric field, nonradiative Auger recombination, and/or surface trap induced recombination. Blinking may be photo-induced or spontaneous, for instance, as described in Stefani et al., “Quantification of photoinduced and spontaneous quantum-dot luminescence blinking.” Physical Review B 72, 125304 (2005), incorporated herein by reference in its entirety for all purposes. Inherent quantum dot blinking is generally believed to interfere with fluorescence quenching assays and techniques are available to limit intermittent fluorescence. In some embodiments herein, labels (such as quantum dots) that blink may be used, for instance, in cases where nucleic acid molecule density on the substrate is high. In examples where two nucleotides with blinking labels or one nucleotide with a blinking label and another with a non-blinking label are incorporated at two nearby spots, signals detected at one or more time points where only one of the two labels is emitting may be used to resolve the two nearby spot locations.

In some embodiments, a subset of nucleic acid molecules (e.g., nucleic acid strands to be sequenced) on the substrate may be active at one or more time points. In some embodiments, at any one time, a first subset of nucleic acid molecules on the substrate is active (e.g., allowing nucleotide incorporation into a sequencing primer using a single-stranded sequence as template) while a second subset of nucleic acid molecules on the substrate is inactive (e.g., not allowing nucleotide incorporation into a sequencing primer using a single-stranded sequence as template). In some embodiments, at one or more time points, a first subset of nucleic acid molecules on the substrate is activated (e.g., by a first set of polymerase and/or primer molecules) for nucleotide incorporation, while a second subset of nucleic acid molecules on the substrate is not activated (e.g., by the first set of polymerase and/or primer molecules), thus only signals associated with the first subset of nucleic acid molecules are detected. At one or more other time points, the second subset of nucleic acid molecules on the substrate is activated (e.g., by a second set of polymerase and/or primer molecules) for nucleotide incorporation, while the first subset of nucleic acid molecules on the substrate is not activated (e.g., by the second set of polymerase and/or primer molecules), thus only signals associated with the second subset of nucleic acid molecules are detected. In some embodiments, the first and second sets of polymerase and/or primer molecules can be introduced at different time points, e.g., in sequential cycles with optional washing steps between cycles (e.g., to remove a set of polymerase and/or primer molecules for SBS of a first subset of strands before introducing the next set of polymerase and/or primer molecules for SBS of a second subset of strands). In some embodiments, regardless of whether a particular strand being sequenced is in the first subset or the second subset, nucleotide incorporation using the particular strand as template can occur in a non-cyclical manner as described herein.

In some embodiments, the substrate can comprise a bead, a planar substrate, a solid surface, a flow cell, a semiconductor chip, a well, a pillar, a chamber, a channel, a through hole, a nanopore, or any combination thereof. In some embodiments, the substrate can comprise a microwell, a micropillar, a microchamber, a microchannel, or any combination thereof.

### D. Signal Deactivation

In some embodiments, one or more of the incorporated nucleotides may be stochastically deactivated (e.g., by photobleaching and/or cleaving the labels) in a non-cyclically manner. In some embodiments, for a given labeled nucleotide, once the label is cleaved or deactivated, the signal intensity (if any remains) associated with the nucleotide no longer changes, e.g., in response to light that bleaches labels on other nucleotides. For instance, in one embodiment, after the fluorescent dye of a particular dye-labeled nucleotide is photobleached (thus fluorescence intensity associated with dye-labeled nucleotide decreases from a first intensity to a second, lower intensity), the photobleached dye-labeled nucleotide does not recover to the first fluorescence intensity. In some embodiments, the fluorescence intensity of the photobleached dye-labeled nucleotide remains at the second intensity which can be zero; in other words, the photobleached dye can go “dark,” e.g., its signal is below a certain threshold or undetectable and does not recover. In some embodiments, an increase in signal intensity due to a nucleotide incorporation event in a method disclosed herein is not detected as an increase due to a photobleached dye recovering from a bleached state. In some embodiments, a photobleached dye herein is prevented from recovering from a bleached state such that an increase in signal intensity is attributable to nucleotide incorporation rather than recovery from photobleaching. In some embodiments, for each label that has been deactivated (e.g., photobleached), the deactivation is complete in that the deactivated label does not recover. In some embodiments, labels at multiple locations (some of which may comprise the same label and others may comprise different labels) are not deactivated (e.g., photobleached) at the same time or in the same time window (e.g., in the same cycle). Rather, in a method disclosed herein, labels at different locations may be deactivated stochastically such that at a given time point or in a given time window, the labels at all locations of the substrate are not completely deactivated whereas for each label the signal deactivation is or will be complete (e.g., no signal recovery from a deactivated state).

In some embodiments where recovery from a deactivated state (e.g., after photobleaching) may occur, a recovery probability may be modeled and used during basecalling. In some embodiments, the recovery probability is modeled using a reference based correction. Dye recovery from photobleaching has been described, for instance, by Braslavsky et al., “Sequence information can be obtained from single DNA molecules,” PNAS 100(7): 3960-64 (2003), incorporated herein by reference in its entirety for all purposes.

In some embodiments, stepwise changes over time in fluorophore emission (e.g., stepwise increases and/or decreases in signal intensity) at the particular spots can be detected and/or monitored. An increase in signal intensity (e.g., due to a nucleotide incorporation) and/or a decrease in signal (e.g., due to a photobleaching event) at a particular spot and in a given time window or time point (e.g., an imaging window in terms of frame/exposure) may partially or completely offset one another. In some embodiments, incorporation of a labeled nucleotide results in an increase in signal intensity characteristic of the label and/or the base of the incorporated labeled nucleotide. For instance, a nucleotide can be labeled with a label having a signal intensity characteristic of the base in that nucleotide, which can be distinguished from the signal intensity of the label on another nucleotide having a different base. In some embodiments, signal deactivation (e.g., by cleaving and/or photobleaching the label) of a labeled nucleotide results in a decrease in signal intensity characteristic of the label and/or the base of the signal-deactivated labeled nucleotide.

### E. Deconvolution and Basecalling

In some embodiments, each type of nucleotide (e.g., nucleotides comprising A, T/U, C, or G) can be labelled with a different fluorophore such that emissions of a particular fluorophore would be passed by one filter and rejected by all others. An exemplary high-throughput sequencing platform for real-time monitoring of biological processes by multicolor single-molecule fluorescence is described in Chen et al., PNAS 111 (2) 664-669 (2014) which is incorporated herein by reference in its entirety for all purposes.

In some embodiments, provided herein is a method comprising the use of labels with differing intensities (e.g., brightness) over a range of wavelengths. When combined with an appropriate filter, different dyes can be registered as different intensities using a single fixed filter and camera. This is advantageous as it results in a simpler and cheaper optical system. Such a labeling scheme may be used in a real-time context (e.g., cycle-less, no terminators) where each nucleotide incorporates and bleaches stochastically. For instance, dyes on incorporated nucleotides may not be completely bleached (or otherwise stochastically removed) before a subsequent nucleotide is incorporated. In some aspects, composition of bases (e.g., contiguous nucleic acid sequences) can be determined in a real-time sequencing approach, where nucleotides incorporate stochastically and labels bleach stochastically. In some embodiments, imaging is continuous in order to observe all incorporation events. In some cases, the average incorporation rate is tuned (e.g., through nucleotide concentration and/or polymerase activity) such that it is unlikely that multiple incorporations occur in a single frame. Similarly, the photobleaching rate can also be tuned (e.g., though laser intensity or oxygen scavenging additives).

While it's possible that photobleaching may occur in any order. incorporation and photobleaching events are matched. Photobleaching can occur with a fixed probability in each time point on the single molecule level. By tracking the incorporation events and photobleaching events, nucleic acid sequences of the strand being synthesized and the complementary template strand can be determined. A Hidden Markov Model (HMM) can be used to deconvolute the detected signal intensities over time in order to detect incorporation and bleaching events.

In some embodiments, the net change in signal intensity at the particular spot and the given time window or time point can be associated with the event(s) at the particular spot, for instance, incorporation of a new labeled nucleotide and photobleaching of one or more already incorporated labeled nucleotides. The one or more already incorporated labeled nucleotides may be at any distance from the newly incorporated labeled nucleotide, e.g., 0, 1, 2, 3, 4, 5, or more nucleotide residues apart. In some embodiments, the net change in signal intensity may be deconvoluted to one or more increases and/or one or more decreases in signal intensity that are characteristic of a nucleotide incorporation event (e.g., incorporation of a nucleotide labeled with a particular fluorophore) and a signal deactivation event (e.g., photobleaching of the same or another particular fluorophore), respectively.

In some embodiments, the deactivating step and/or the detecting step can be carried out as detectably labeled nucleotides are continuously provided to contact the nucleic acid molecule and/or the primer. In some embodiments, the detecting step is performed in real time as the nucleotide incorporation and signal deactivation (e.g., photobleaching) events occur. In some embodiments, the detecting step is not carried out using multiple switchable optical filters each for detecting a different detectable label. In some embodiments, the detecting step can be carried out using a dichroic filter to split optical signals into channels for detecting a different detectable label in each channel. In some embodiments, the detecting step can be carried out using total internal reflection fluorescence (TIRF) microscopy. In some embodiments, the signals in the detecting step can be compensated for background signal.

In some embodiments, nucleotide identification using the time trace can comprise probabilistically identifying the first, second, third, and/or fourth detectably labeled nucleotides. In some embodiments, the probabilistically identifying step can comprise assigning a state of signal intensity to each detectable label and decoding the time trace. In some embodiments, the state of signal intensity corresponds to a fixed value of signal intensity (e.g., sum of relative fluorescence over a range of excitation wavelengths). In some embodiments, the state of signal intensity corresponds to a range of signal intensities. In some embodiments, the state of signal intensity corresponds to a Gaussian distribution of signal intensities. In some embodiments, decoding the time trace may comprise pairing an incorporation event with a deactivation event of the detectable label of the nucleotide incorporated in the incorporation event. In some embodiments, decoding the time trace may comprise using a transition probability between two states of signal intensity, and the transition may comprise an incorporation event, a deactivation event (e.g., photobleaching), or an incorporation event and a deactivation event of the same label or different labels at a substrate location. In some embodiments, the transition probability between two states of signal intensity is fixed. In some embodiments, the transition probability between two states of signal intensity is fitted.

In some embodiments, a Hidden Markov Model (HMM) can be used to analyze the incorporation event(s) and/or the deactivation event(s) at one or more substrate locations by observing states of signal intensity and transitions between the states. In some embodiments, using the HMM comprises providing transition probabilities between states of signal intensity due to nucleotide incorporations and label bleaching where individual label bleaching is not expected to recover. For instance, the HMM can model a first state with two currently unbleached labels emitting, one on the incorporated first detectably labeled nucleotide and the other on the incorporated second detectably labeled nucleotide. In this example, the first state may transition into a second state where the label on the incorporated first detectably labeled nucleotide is bleached, or into a third state where the label on the incorporated second detectably labeled nucleotide is bleached. The first state may also transition into a fourth state due to incorporation of a third detectably labeled nucleotide, while the labels on the incorporated first and second detectably labeled nucleotides are not bleached. In some embodiments, decoding the time trace may comprise using the Viterbi algorithm for the HMM that represents incorporation and deactivation events.

In some embodiments, one or more of the sequence reads are about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. Mapping of the sequence reads can be achieved by comparing the sequence of the reads with the sequence of the reference to determine the origin of the sequenced nucleic acid molecule (e.g., from a virus such as a coronavirus, e.g., SARS-COV-2). In some embodiments, the sequence reads can be mapped to one or more reference sequences or genomes. For instance, sequence reads generated using a method disclosed here for sequencing-based SARS-COV-2 detection in a sample may map preferentially to a SARS-COV-2 reference sequence or genome over a background of human sequences and other viral sequences. In some embodiments, certain degrees of mismatch (e.g., 0-2 mismatches per read, 2-5 mismatches per read, or 5 or more mismatches per read) may be allowed, and permitted degree of mismatch may be selected and/or adjusted depending on the application. In some embodiments, the degree of mismatch may be used to account for minor polymorphisms that may exist between the reference sequence or genome and the nucleic acid sequences in a mixed sample. In some embodiments, the degree of mismatch may be used to account for sequencing errors, e.g., technical errors rather than real differences in the sequence (e.g., sequence differences from two copies of a similar sequence in a sample). For instance, errors may be introduced in the manipulation of nucleic acids prior to or during single molecule sequencing reactions and/or may be introduced due to the intrinsic error rate of the polymerase used in the reactions.

In some embodiments, one or more of the sequence reads are no more than 100, no more than 90, no more than 80, no more than 70, no more than 60, no more than 50, no more than 40, no more than 30, no more than 20, no more than 15, or no more than 10 nucleotides in length. In some embodiments, the determined sequence of the nucleic acid molecule may be about 8, about 12, about 16, about 20, about 24, about 28, about 32, about 36, or about 40 nucleotides in length. In some embodiments, the determined sequence of the nucleic acid molecule may be between about 5 and about 50 nucleotides in length, such as between about 10 and about 35 nucleotides in length, or between about 15 and about 30 nucleotides in length.

In some embodiments, the methods described herein further comprise reporting information determined using the analytical methods and/or generating a report containing the information determined suing the analytical methods. For example, in some embodiments, the method further comprises reporting or generating a report containing related to the identification of a variant in a polynucleotide derived from a subject (e.g., from a virus that has infected the subject or within a subject's genome). Reported information or information within the report may be associated with sequencing reads mapped to a reference sequence, a detected variant (such as a detected structural variant or detected SNP or a sequence variant in a viral genome), one or more assembled consensus sequences and/or the a validation statistic for the one or more assembled consensus sequences. The report may be distributed to or the information may be reported to a recipient, for example a clinician, the subject, or a researcher.

### IV. Optical Systems

In some embodiments, provided herein is a total internal reflection fluorescence (TIRF) imaging system (e.g., a system for TIRF microscopy), and a method for using the TIRF imaging system for detecting and processing optical signals for nucleic acid (e.g., DNA or RNA) sequencing.

In some embodiments, provided herein is a cheap and simple TIRF imaging system for use in a user facing analytical equipment, e.g., for nucleic acid sequencing. Existing TIRF platforms either use objective style TIRF optics (which is expensive, and typically requires immersion oil between the lens and the substrate such as a cover slip, e.g., a cover glass) or prism style TIRF optics (which usually require low autofluorescence fused silica prisms, and immersion/optical matching oil between the substrate and the prism). In some embodiments, a prism-style TIRF platform is attractive because cheaper low numerical aperture (NA) objective lens can be used. Numerical Aperture (also termed Object-Side Aperture) is a value for microscope objectives and condensers: NA=n×sin (μ) or n×sin (α), where n represents the refractive index of the medium between the objective front lens and the specimen, and μ or α is the one-half angular aperture of the objective. The numerical aperture of a microscope objective is a measure of its ability to gather light and resolve fine specimen detail at a fixed object distance.

However, in some embodiments, in a user facing device dealing with oil on the prism is messy and inconvenient. To address these issues, in some embodiments, the prism is embedded in the substrate. In some embodiments, the prism is used as the substrate, making this component disposable, but where fused silica prisms are used this is cost prohibitive. In some embodiments, the fused silica prism can be replaced with a low auto-florescence plastic, for example ZEONEX 5000*. A plastic may be chosen to show minimal auto-florescence for a give excitation wavelength. In some embodiments, the prism comprises one or more optical quality plastic materials with a low autofluorescence, for use in detection by fluorescence and laser induced fluorescence techniques. For example, PDMS shows a comparatively low auto-florescence compared to other common plastics and can be used as a prism in a TIRF imaging system disclosed herein. In some embodiments, the prism comprises one or more commercially available plastic chip materials, such as PMMA, COC, PC, and/or PDMS. See, e.g., Piruska et al., “The autofluorescence of plastic materials and chips measured under laser irradiation,” Lab Chip, 2005, 5, 1348-1354, incorporated herein by reference in its entirety for all purposes.

In some embodiments, a plastic prism may form part of a disposable flowcell or flowcell/reagent cartridge. In some embodiments, the prism surface itself may be used as a substrate for the attachment of analytes to be imaged. In some embodiments, the prism may be bonded to a substrate.

In some embodiments, in order to reduce the effect of auto-florescence, an excitation filter may be used below and/or above the substrate. In some embodiments, the excitation filter is selected such that it passes the excitation wavelength and blocks auto-florescence. In some embodiments, the excitation filter blocks at least about 10%, at least about 20%, at least about 30%, at least about 40%, at least about 50%, at least about 60%, at least about 70%, at least about 80%, at least about 90%, or about 100% auto-florescence. Alternatively or in addition to the excitation filter, an additive may be added to the prism plastic to act as a filter. In some embodiments, a system disclosed herein can be used in combination with an emission filter.

In some embodiments, a lightguide style TIRF may be used. In some embodiments, a lightguide is integrated into a flowcell.

Systems as described above may be of utility in diagnostic or life science equipment and elsewhere. In some embodiments, an optical system disclosed herein may form part of a DNA or RNA sequencing instrument. In some embodiments, an optical system disclosed herein may be incorporated into a DNA or RNA sequencing system where a low cost optical approach to single molecule imaging is desirable. In some embodiments, a TIRF prism may be incorporated into a disposable cartridge or flowcell.

### V. Methods for Low Cost Nucleic Acid Sequencing

In some embodiments provided herein, methods and compositions enabling low cost nucleic acid (e.g., DNA and/or RNA) sequencing are provided. In some embodiments, provided herein are improvements relating to simplification of the fluidic system in a sequencing-by-synthesis (SBS) style sequencing platform. In some embodiments, the sequencing platform may be single molecule or use ensemble methods (e.g., sequencing involving clusters, polymerization, polony, or nano-balls, etc.). In some embodiments, the fluidic system described may also be used in other applications where it is desirable to control the motion of reagents, for example, to control the movement of a nucleotide from a first region to a second region where it is incorporated by a polymerase.

### A. Fluidics

In some embodiments, it is desirable to use flowcell integrated reagents or reagents closely coupled to the flowcell. In some embodiments, reagents are included in a flowcell or a cartridge system. In some embodiments, reagents (for example nucleotides, e.g., modified nucleotides and/or unmodified nucleotides) are confined in a starting region (e.g., in a flowcell or a cartridge system) and transported to another region, e.g., in the flowcell or the cartridge system. In some embodiments, electrophoretic flow, diffusion, charge based methods, or pressure may be used to move reagents (including nucleotides) from one region to another, e.g., in a flowcell or a cartridge system.

### B. Charge Barriers

In some embodiments, the reagents may be initially confined by a charge barrier (e.g., electric field). For example, one or more negative charged reagents (e.g., one or more nucleotides) may be confined to a cavity/region using a negatively charged electrode, or may be attracted to a cavity/region using a positively charged electrode. In some embodiments, a powered electrode, a charged surface (e.g., a statically charged surface), or barrier may be used instead of or in combination with an electrode.

In some embodiments, the charge may be used to release a reagent. For example, in the case of a negatively charged reagent (e.g., a nucleotide), a positively charged electrode (which attracts the negatively charged reagent) can be made more negative (e.g., charge neutral or more negatively charged) to allow the reagent (e.g., nucleotide) to diffuse away from the electrode. For instance, a nucleotide can be allowed to freely diffuse from a first region (e.g., a reagent chamber where the electrode is) into a second region (e.g., a region where a nucleotide is incorporated by a polymerase, such as a main chamber).

In some embodiments, either alternatively, or in combination with any of the preceding embodiments, a charged electrode may act as a barrier to prevent a reagent (e.g., a nucleotide) in a first region from entering a second region. For example, a negatively charged reagent (e.g., nucleotide) may be repelled by a negatively charged electrode (e.g., in a first chamber such as a reagent chamber) to prevent it from diffusing (e.g., into a second chamber such as a main chamber). In some embodiments, the barrier charge may be removed (e.g., made more positive), allowing the reagent (e.g., nucleotide) to diffuse from a first region into a second region (e.g., a region where a nucleotide is incorporated by a polymerase, such as a main chamber).

In some embodiments, in combination with any of the preceding embodiments, an additional electrode may be used to assist the motion of the reagent (c.g., nucleotide). In some embodiments, the additional electrode may reside in the second region (e.g., a region where a nucleotide is incorporated by a polymerase). In some embodiments, the additional electrode may be charged to attract the reagent (e.g., nucleotide) toward the second region (e.g., the additional electrode can be positively charged to attract one or more nucleotides).

In some embodiments, an electrode may comprise a conductive material such as indium tin oxide (ITO). In some embodiments, electrodes may be formed using an indium tin oxide (ITO) coating or using any suitable conductive material. In some embodiments, the electrodes may be formed using a material deposited by vapor deposition or another method. In some embodiments, other materials which may be selectively statically or dynamically charged are used to generate electrodes, and a coating of the material may be on a glass, fused silica, or any other surface. In some embodiments, an electrode disclosed herein may comprise a glass or fused silica. For instance, a glass electrode can be used to provide an ion-selective electrode made of a doped glass membrane that is sensitive to an ion. In some embodiments, the electrodes may be electrically charged or discharged, or mechanically inserted and/or removed (e.g., in a region, such as a reagent chamber and/or in a main chamber).

In some embodiments, the charge-based methods using electrodes may be used to confine a reagent (e.g., nucleotide) until it is desirable to expose the reagent, e.g., to a second region (e.g., an incorporation region). In some embodiments, the electrodes are used to provide reagent confinement regions, where charged reagents are confined until the charge on the electrode is changed to release and/or repel the reagents. In some embodiments, multiple reagent confinement regions may be used to expose multiple reagents to one or more other regions (e.g., incorporation regions).

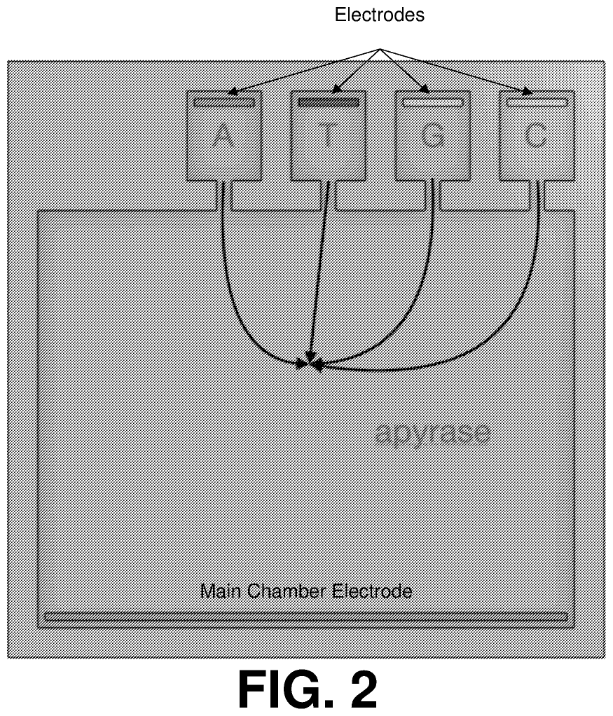

For example, multiple reagent confinement regions may confine different nucleotide types. For example (see e.g., FIG. 2), four regions each containing one of guanine (unmodified and/or modified guanine nucleotides), adenine (unmodified and/or modified adenine nucleotides), cytosine (unmodified and/or modified cytosine nucleotides), and thymine (unmodified and/or modified thymine nucleotides). In some embodiments, the reagent confinement region for each of these nucleotides may sequentially (or in combination) be exposed to a second region, such as an incorporation region in a main chamber.

In some embodiments, a bead comprising one or more reagents immobilized thereon can be moved under charge. In some embodiments, reagents may include a bead, wherein one or more reagents are confined on the surface of the bead. The bead itself and/or the reagent(s) thereon can be charged, allowing the bead to be moved under charge. In some embodiments, conditions in a second region may be such that reagents attached to the bead surface can be released from the bead (e.g., from the bead surface) in the second region.

In some embodiments, the approaches disclosed herein for reagent transport may be used to enable a fluidic system from a sequencing-by-synthesis platform (FIG. 2 shows an exemplary configuration). In some embodiments, labelled (or unlabeled) nucleotides can be sequentially exposed to template(s) under synthesis. The incorporation of each nucleotide type can be sequentially detected.

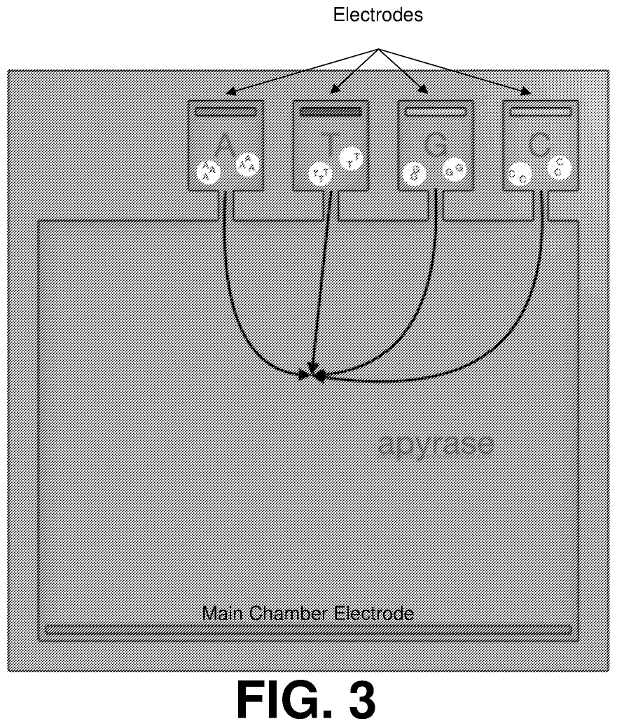

In some embodiments, the reagents (e.g., nucleotides) may be confined in reagent “bubbles”. These bubbles may be formed from a confinement layer surrounding a reagent. For example, this layer maybe a lipid bilayer. Charge may be used to apply pressure to these reagent bubbles, breaking the layer and releasing the enclosed reagents. Charge may also be used to otherwise disrupt the stability of the layer (for example, the stability of a lipid bilayer). See FIG. 3 for an example.

In some embodiments, heat may be used (e.g., as an alternative or in combination with any of the preceding embodiments) to break the reagent bubble, releasing reagents. Said released reagents (e.g., nucleotides) are then free to diffuse into a second region and/or to be pulled under a charge.

In some embodiments, a change in pH may be used (e.g., as an alternative or in combination with any of the preceding embodiments) to break the reagent bubble. In some embodiments, a surfactant may be used (e.g., as an alternative or in combination with any of the preceding embodiments) to break the reagent bubble. In some embodiments, a lysis solution may be used (e.g., as an alternative or in combination with any of the preceding embodiments) to break the reagent bubble. In some embodiments, SDS may be used (e.g., as an alternative or in combination with any of the preceding embodiments) to break the reagent bubble. In some embodiments, an enzyme may be used (e.g., as an alternative or in combination with any of the preceding embodiments) to break the reagent bubble. The bubble maybe broken enzymatically. For example, alpha hemolysin nanopores, or other nanopores may introduce instability and break a lipid bubble, and can be used in a method disclosed herein.

In some embodiments, the reagent bubble maybe transported under pressure from one region to a second region. A surface charge maybe introduced to transport or confine the bubble under charge using methods described herein in “charge barriers.” Reagents maybe transported to a second region of a different temperature, a different pH, and/or containing other reagents (for example, one or more enzymes), in order to break the reagent bubble.

In some embodiments, the layer (for example a lipid bilayer) may incorporate photo-sensitive components. Light (UV, visible or other wavelengths) may be used to disrupt these photo-sensitive components and break the bubble, releasing the enclosed reagents.

In some embodiments, a bubble based confinement system may be used to sequentially expose an incorporation region to nucleotides (or other reagents) in charge barriers, as described herein.

### C. Physical Confinement in Externally Activated Chambers

In some embodiments, reagents (e.g. nucleotides) may be confined in chambers. In some embodiments, these chambers may have an externally activated valve or “door”.

In some embodiments, external pressure may be used to apply pressure to a valve embedded in a flowcell or a cartridge system to allow reagents to move under pressure, or diffuse, or under another force (e.g., electric charge), to a second chamber (e.g., an incorporation chamber and/or an imaging chamber). The incorporation chamber and the imaging chamber can be the same or different.

In some embodiments, heat may be applied to selectively open reagent chambers. In some embodiments, heat activation to selectively open regent chambers may be via expansion or contraction of a material forming the valve door.

In some embodiments, any one or more chamber valves can be photo-activated. In some embodiments, the valve material is deformed when exposed to light (e.g., light of a specific wavelength(s)).

### D. Magnetic and Optical Tweezer Reagent Steering

In some embodiments, optical tweezers may be used to steer reagents from one region of the flowcell or cartridge to another along a path, such as along a fixed or flexible path. In some embodiments, the path may be a fixed path created using a series of LEDs focused on the desired reagent path. In some embodiments, optical tweezers may be used to steer reagents, beads attached to reagents, and/or reagent bubbles.

In some embodiments, reagents may be confined on magnetic beads, which may be manipulated under a magnetic field. Magnetic beads can be steered from one region of the flowcell or cartridge to another along a path, such as along a fixed or flexible path.

### E. Homopolymer Run Length Determination

In some embodiments, determining the length of homopolymer runs in sequencing-by-synthesis is a problem of interest that can be addressed using a method disclosed herein. In some embodiments, determining the length is often solved through the use of reversible terminators. In some embodiments, the terminators prevent multiple nucleotides of the same base type from incorporating.

In some embodiments, provided herein are methods that provide an approach for use in sequencing-by-synthesis, such as for single molecule sequencing, e.g., a sequencing-by-synthesis technology based on real-time imaging of fluorescently tagged nucleotides as they are synthesized along individual nucleic acid template molecules. Exemplary sequencing-by-synthesis platforms include those described in U.S. Pat. No. 7,056,661, PCT/US2022/034346 (published as WO 2022/271701), US 2010/0227327 A1, and PCT/US2023/062148, all of which are herein incorporated by reference in their entireties for all purposes.

In some embodiments, multiple fluorescently labelled nucleotides are incorporated into a homopolymer under synthesis. In some embodiments, each incorporated nucleotide causes a stepwise increase in fluorescent intensity. In some embodiments, the initial intensity provides an estimate of the number of homopolymers incorporated. In some embodiments, step-counting (e.g., step-counting-photo-bleaching described in Mira et al., “Counting the Number of Fluorophores Labeled in Biomolecules by Observing the Fluorescence-Intensity Transient of a Single Molecule,” BCSJ 78(9): 1612-18 (2005), incorporated herein by reference in its entirety for all purposes) can be used to provide additional information on the number of nucleotides incorporated.

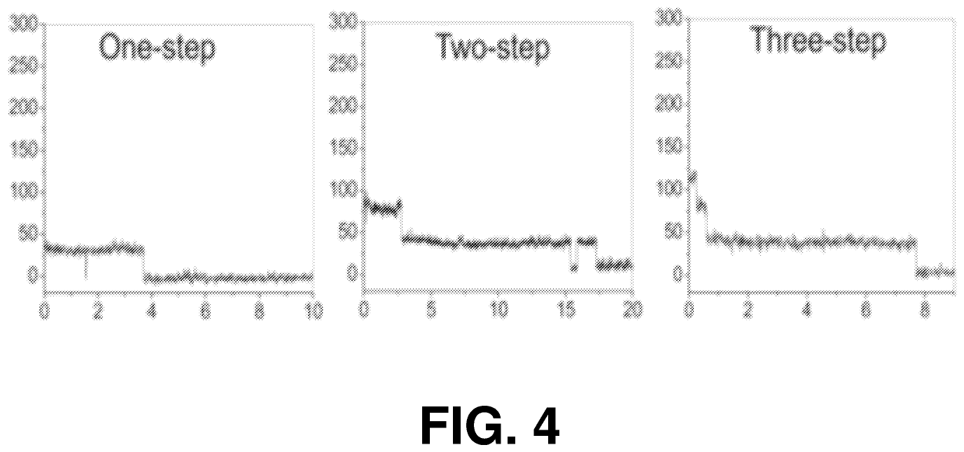

In some embodiments, by reducing or extinguishing signals associated with the nucleotides (e.g., by bleaching the fluorescent labels), the signal from the labelled nucleotides may be removed from the signals presented by the template (e.g., strand undergoing sequencing), for instance, in a step-wise manner. In some embodiments, the bleaching of the nucleotide label removes the requirement to cleave labels and simplifies analysis. In an exemplary method, plots of fluorescence intensity versus time that show one, two, three, or more steps in photobleaching can be generated (e.g., as shown in FIG. 4). The intensity can be plotted in arbitrary units, and empirical histogram showing the distribution frequency of detected spots can be grouped by steps of quantized photobleaching. Statistic model can be used to fit empirical histogram of photobleaching steps, and the empirical data can be compared with model predictions.

In some embodiments, stepwise changes over time in fluorophore emission (e.g., stepwise increases and/or decreases in signal intensity) at the particular spots can be detected and/or monitored. An increase in signal intensity (e.g., due to a nucleotide incorporation) and/or a decrease in signal (e.g., due to a photobleaching event) at a particular spot and in a given time window or time point (e.g., an imaging window in terms of frame/exposure) may partially or completely offset one another. In some embodiments, incorporation of a labeled nucleotide results in an increase in signal intensity characteristic of the label and/or the base of the incorporated labeled nucleotide. For instance, a nucleotide can be labeled with a label having a signal intensity characteristic of the base in that nucleotide, which can be distinguished from the signal intensity of the label on another nucleotide having a different base. In some embodiments, signal deactivation (e.g., by cleaving and/or photobleaching the label) of a labeled nucleotide results in a decrease in signal intensity characteristic of the label and/or the base of the signal-deactivated labeled nucleotide.

In some embodiments, the net change in signal intensity at the particular spot and the given time window or time point can be associated with the event(s) at the particular spot, for instance, incorporation of a new labeled nucleotide and photobleaching of one or more already incorporated labeled nucleotides. The one or more already incorporated labeled nucleotides may be at any distance from the newly incorporated labeled nucleotide, e.g., 0, 1, 2, 3, 4, 5, or more nucleotide residues apart. In some embodiments, the net change in signal intensity may be deconvoluted to one or more increases and/or one or more decreases in signal intensity that are characteristic of a nucleotide incorporation event (e.g., incorporation of a nucleotide labeled with a particular fluorophore) and a signal deactivation event (e.g., photobleaching of the same or another particular fluorophore), respectively.

### F. Surface Attachment

Provided herein are a number of methods to be used to attach strands undergoing sequencing to the surface of a flowcell and/or sequencing region. In some embodiments, strands may be noncovalently bound to a surface to be synthesized to a surface using a charge based interaction, for example, as in Belosludtsev et al., “DNA Microarrays Based on Noncovalent Oligonucleotide Attachment and Hybridization in Two Dimensions,” Analytical Biochemistry 292(2): 250-256 (2001), incorporated herein by reference in its entirety for all purposes. In some embodiments, the strands after being noncovalently bound may then undergo single molecule sequencing-by-synthesis. In some embodiments, sequencing-by-synthesis may use targeted primers and/or random primers.

In some embodiments, a surface attachment procedure where single molecule probes noncovalently attached to a surface using a charge based interaction are used to capture templates from solution. Probes may be targeted or random.

In some embodiments, provided herein is a fluidic/reagent distribution system using charge to confine and direct one or more reagents. In some embodiments, the charge on a reagent is used to attract a reagent from a first region to a second region. In some embodiments, the charge on a reagent is used to confine the reagent in a first region, and removed to release it to a second region. In some embodiments, the charge on a reagent is used to attract a reagent from a first region to a second region, and the charge on the reagent is used to confine it in a first region and removed to release it to a second region.

In some embodiments, the reagent is a nucleotide. In some embodiments, the charge is created using one or more electrodes. In some embodiments, the charge is a static charge. In some embodiments, the charge is a combination of static charges and dynamic charges created by one or more electrodes.

In some embodiments, reagents are digested (for example nucleotides by apyrase, which can catalyze the hydrolysis of NTP/dNTP to yield NMP/dNMP and inorganic phosphate) at the second region. In some embodiments, reagents are removed from the second region using any of the methods described herein.

In some embodiments, provided herein is a reagent distribution system where reagents are confined with reagent bubbles (vesicles or vesicles-like structures), and where reagents confined within the bubbles may be selectively released.

In some embodiments, the bubble is a lipid bilayer. In some embodiments, the bubble is a lipid bilayer, and application of a lysis buffer is used to release reagents. In some embodiments, the bubble is a lipid bilayer, where heat is used to cause bilayer instability and release reagents. In some embodiments, the bubble is a lipid bilayer, where an electric charge, or electric field is used to cause bilayer instability and release reagents. In some embodiments, the bubble is a lipid bilayer, where a change is pH is used to cause bilayer instability and release reagents. In some embodiments, the bubble is a lipid bilayer, where a surfactant is used to cause bilayer instability and release reagents. In some embodiments, the bubble is a lipid bilayer, where an enzyme is used to cause bilayer instability and release reagents. In some embodiments, the bubble is a lipid bilayer, where a nanopore, for example alpha hemolysin, is used to cause bilayer instability and release reagents.

In some embodiments, reagent bubbles are directed under charge/electric fields.

In some embodiments, provided herein is a method whereby reagents are physically confined in chambers, with reagent valve which may be opened (and optionally closed) to release reagents.

In some embodiments, heat is used to deform and open the reagent chamber valve. In some embodiments, pH is used to deform and open the reagent chamber valve. In some embodiments, light is used to deform and open the reagent chamber valve. In some embodiments, pressure is externally applied to open the reagent chamber valve.

In some embodiments, provided herein is a method where optical tweezer are used to steer reagents from a confinement region to a second region. Provided herein is a method where a magnetic field is used to steer reagents confined on magnetic beads.

In some embodiments, reagents are further confined on beads or in vesicles (bubbles), steered using optical tweezers.

In some embodiments, reagents are moved to a region with conditions such that reagents are released from the bead/vesicle. In some embodiments, reagents are moved to a region with conditions such that reagents are released from the bead.

In some embodiments, provided herein is a method where the length of a homopolymer run is determined by the fluorescence intensity of multiple incorporations.

In some embodiments, nucleotides undergo photobleaching. In some embodiments, photobleaching steps are used to help determine the number of nucleotides present. In some embodiments, sufficient time is allowed for complete bleaching. In some embodiments, the bleaching process is monitored to ensure that all labels are fully bleached.

In some embodiments, provided herein is a method where single stranded DNA is attached to a surface noncovalently using a charge-based interaction.

In some embodiments, the strand is a DNA fragment to be sequenced. In some embodiments, the strand is randomly primed to enable sequencing-by-synthesis. In some embodiments, the strand is primed using a targeted primer to enable sequencing-by-synthesis.

In some embodiments, DNA probes are attached to a surface, to capture strands to be sequenced using sequencing-by-synthesis. In some embodiments, the DNA probes attached to the surface are random probes. In some embodiments, the DNA probes attached to the surface are targeted probes.

### VI. Compositions, Kits, and Applications

Also provided herein are compositions and kits comprising one or more of the primers, nucleic acid molecules, substrates, nucleotides including detectably labeled nucleotides, polymerases, and reagents for performing the methods provided herein, for example reagents required for one or more steps comprising hybridization, ligation, amplification, detection, sequencing, and/or sample preparation as described herein, for example, in Section III.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for sample processing, such as nucleic acid extraction, isolation, and/or purification, e.g., RNA extraction, isolation, and/or purification. In some embodiments, the kits contain reagents, such as enzymes and buffers for ligation and/or amplification, such as ligases and/or polymerases. In some embodiments, the kits contain reagents, such as enzymes and buffers for primer extension and/or nucleic acid sequencing. such as polymerases and/or transcriptases. In some aspects, the kit can also comprise any of the reagents described herein, e.g., buffer components for tuning the rate of nucleotide incorporation and/or for tuning the rate of signal deactivation (e.g., by photobleaching). In some embodiments, the kits contain reagents for signal detection during sequencing, such as detectable labels and detectably labeled molecules. In some embodiments, the kits optionally contain other components, for example nucleic acid primers, enzymes and reagents, buffers, nucleotides, modified nucleotides, and reagents for additional assays.

In some aspects, the provided embodiments can be applied in analyzing nucleic acid sequences, such as DNA and/or RNA sequencing, for example single molecule real-time DNA and/or RNA sequencing. In some aspects, the embodiments can be applied in an imaging or detection method for multiplexed nucleic acid analysis. In some aspects, the provided embodiments can be used to identify or detect regions of interest in target nucleic acids, such as viral DNA or RNA. In some embodiments, the region of interest comprises one or more nucleotide residues, such as a single-nucleotide polymorphism (SNP), a single-nucleotide variant (SNV), substitutions such as a single-nucleotide substitution, mutations such as a point mutation, insertions such as a single-nucleotide insertion, deletions such as a single-nucleotide deletion, translocations, inversions, duplications, and/or other sequences of interest.

In some aspects, the embodiments can be applied in investigative and/or diagnostic applications, for example, for characterization or assessment of a sample from a subject. Applications of the provided method can comprise biomedical research and clinical diagnostics. For example, in biomedical research, applications comprise, but are not limited to, genetic and genomic analysis for biological investigation or drug screening. In clinical diagnostics, applications comprise, but are not limited to, detecting gene markers such as disease, immune responses, bacterial or viral DNA/RNA for patient samples, loss of genetic heterozygosity, the presence of gene alleles indicative of a predisposition towards disease or good health, likelihood of responsiveness to therapy, or in personalized medicine or ancestry.

### VII. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

The terms “detectable label.” “optical label,” and “label” are used interchangeably herein to refer to a directly or indirectly detectable moiety that is coupled to or may be coupled to another moiety, for example, a nucleotide or nucleotide analog. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

In some embodiments, a detectable label is or includes a fluorophore. Exemplary fluorophores include, but are not limited to, fluorescent nanocrystals; quantum dots; d-Rhodamine acceptor dyes including dichloro[R110], dichloro[R6G]. dichloro[TAMRA], dichloro[ROX] or the like; fluorescein donor dye including fluorescein, 6-FAM, or the like; Cyanine dyes such as Cy3B; Alexa dyes, SETA dyes, Atto dyes such as atto 647N which forms a FRET pair with Cy3B and the like. Fluorophores include, but are not limited to, MDCC (7-diethylamino-3-[([(2-maleimidyl)ethyl]amino)carbonyl]coumarin), TET, HEX, Cy3, TMR, ROX, Texas Red, Cy5, LC red 705 and LC red 640.

In some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label.

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. Enzymatic extension can be performed by an enzyme including, but not limited to, a polymerase and/or a reverse transcriptase. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence can be used as a template for single-strand synthesis of a corresponding cDNA molecule. Furthermore, a poly (dT) sequence may be used as a sequencing primer for sequencing RNA molecules comprising poly(A) tails.

A “non-terminating nucleotide” or “incorporating nucleotide” can include a nucleic acid moiety that can be attached to a 3′ end of a polynucleotide using a polymerase or transcriptase, and that can have another non-terminating nucleic acid attached to it using a polymerase or transcriptase without the need to remove a protecting group or reversible terminator from the nucleotide. Naturally occurring nucleic acids are a type of non-terminating nucleic acid. Non-terminating nucleic acids may be labeled or unlabeled.

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but are typically at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In some embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

In some embodiments, PCR amplification can include reactions such as, but not limited to, a strand-displacement amplification reaction, a rolling circle amplification reaction (e.g., the multiple repeats can be cleaved from the rolling circle amplification product), a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

Suitable examples of DNA polymerases that can be used include, but are not limited to: E.coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g., M-MLV, and compositions including unmodified and modified enzymes are commercially available, e.g., ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g., an AMV or MMLV reverse transcriptase.

### EXAMPLES

The following examples are included for illustrative purposes only and are not intended to limit the scope of the present disclosure.

### Example 1: Error Rate Reduction in Double Stranded Nucleic Acid Sequencing

Double stranded nucleic acid fragments, such as DNA fragments, are attached weakly to a surface. The double stranded nucleic acid fragments are melted or denatured to become a first single stranded nucleic acid and a second single stranded nucleic acid. The first single stranded nucleic acid and the second single stranded nucleic acid are attached to the surface by use of probes which bind to the nucleic acids or electrical fields. Diffusion of the first single stranded nucleic acid and the second single stranded nucleic acid across the surface is limited by use of electrical fields. In one particular example, the first single stranded nucleic acid and the second single stranded nucleic acid are attached by use of positive electric charges on the surface, which attract the negatively charged nucleic acids, and diffusion is limited by using negatively charged electrodes to generate negatively charged electric fields between where the double stranded nucleic acid fragments bound, leading the first single stranded nucleic acid and the second single stranded nucleic acid to be in closer proximity to each other than to other single stranded nucleic acids generated from different double stranded nucleic acids.

Clusters are formed from the first single stranded nucleic acid and the second single stranded nucleic acid. The clusters are used to spatially sequence the first single stranded nucleic acid and the second single stranded nucleic acid. For example, the spatial sequencing may be done by sequencing-by-synthesis methods. During the image analysis portion of the sequencing, locations on the surface are recorded. Post-basecalling or as part of the basecalling process, an algorithm is used to determine if two nearby templates came from the same nucleic acid, for example using Euclidean distance or other methods. If two strands are determined to come from the same source double stranded nucleic acid fragment, the strand sequences are combined.

When sequences from each strand are combined, basses that do not match are treated in a number of ways. The bases may be replaced with “N”s if the mismatch is not resolved. Alternatively, base quality scores from each strand may be used to select which basecall is most accurate. Features extracted during image analysis are used to influence base quality scores, such as circularity of the cluster in the position of the likely error, local background, or other features that can cause errors. Errors in each strand likely have different error characteristics due to the nature of phasing errors, causing early bases to be of higher quality. When sequences from each strand are combined, early bases from each strand are given preference to later bases sequenced in the complementary strand, allowing for more accurate basecalling. This approach allows for more accurate basecalling while increasing effective read length. Where strands are longer than the platform read length but short enough that reads from the first single stranded nucleic acid and the second single stranded nucleic acid overlap, the overlap is used to assemble the two reads into a longer read. This process may be used to clusters formed from the forward and revers strands that are not well separated and would normally be termed mixed where a hidden Markov model or other algorithm is used to extract the most likely source template from the convolved signal of the forward and reverse strands.

### Example 2: Single Cell Spatial Sequencing

Single cells are isolated and attached to a surface. The cells are attached through a charge-based attachment or they are attached using monoclonal or polyclonal antibody or any other method for attaching cells to a surface. The attachment sites for the single cells are spread across the surface, either randomly or as a pattern. Attachment sites are of a size that only a single cell can attach to each attachment site. Attachment sites contain a reverse transcriptase for production of cDNA.

Once the single cells are attached, the single cells are lysed, extracting nucleic acids from the single cells. Cells are lysed using any method (e.g., SDS, lysis buffer, toxins, heat, pH, ect.). Once cells are lysed, the reverse transcriptase bound to the surface at the attachment site converts extracted RNA to cDNA.

Electric charges are used as charge barriers around the cell to confine extracted nucleic acids and cDNA to a region near the cell, such as the attachment site. Charge barriers include negatively charged patterned electrodes which repel negatively charged nucleic acids and prevent them from diffusing and a positive electrode used to pull down the negatively charged nucleic acids to confine them to the attachment site. Once the nucleic acid is confined on the surface, index sequences are introduced to the confined fragments using ligation before spatial sequencing is performed on the nucleic acids.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method for nucleic acid sequencing, comprising:
a) localizing a double stranded nucleic acid to a location on a substrate;
b) generating a first single stranded nucleic acid and a second single stranded nucleic acid from the localized double stranded nucleic acid;
c) restricting diffusion of at least one member selected from the group consisting of the first single stranded nucleic acid and the second single stranded nucleic acid;
d) attaching the first and second single stranded nucleic acids at sites near the location on the substrate;
e) obtaining sequencing reads from the attached first single stranded nucleic acid and sequencing reads from the attached second single stranded nucleic acid; and
f) associating a sequence of the first single stranded nucleic acid with a sequence of the second single stranded nucleic acid, thereby determining a sequence of the double stranded nucleic acid.

2. The method of claim 1, wherein the localizing in a) comprises non-covalently attaching the double stranded nucleic acid to the location on the substrate.

3. (canceled)

4. The method of claim 1, wherein the localizing in a) comprises hybridizing a region of the double stranded nucleic acid to a nucleic acid probe immobilized directly or indirectly on the substrate.

5. The method of any one of claim 1, wherein the double stranded nucleic acid is from a cell or tissue sample.

6. The method of claim 1, wherein the double stranded nucleic acid is a fragmented DNA.

7. The method of claim 1, wherein the double stranded nucleic acid is an amplification product of a cellular DNA or RNA or cell-free DNA.

8.-13. (canceled)

14. The method of claim 1, wherein restricting diffusion of the first and second single stranded nucleic acids in c) comprises capturing the first and second single stranded nucleic acids by nucleic acid probes immobilized directly or indirectly on the substrate.

15.-16. (canceled)

17. The method of claim 1, wherein at least one of the following holds:
in d), the first and second single stranded nucleic acids are covalently attached at the sites near the location on the substrate; or
in d), the first and second single stranded nucleic acids are noncovalently attached at the sites near the location on the substrate.

18. The method of claim 1, wherein in d), the sites are no more than about 8 μm from the location.

19. (canceled)

20. The method of claim 18, wherein both sites are no more than about 2 μm from the location.

21. (canceled)

22. The method of claim 1, wherein the attached first single stranded nucleic acid and the attached second single stranded nucleic acid are clonally amplified to form clusters of amplicons on the substrate.

23.-28. (canceled)

29. The method of claim 1, wherein optical signals at one of the sites are optically resolvable from optical signals at the other site.

30.-31. (canceled)

32. The method of claim 1, wherein the sequence of the first single stranded nucleic acid and the sequence of the second single stranded nucleic acid are determined independently of one another.

33. The method of claim 1, comprising:
comparing the sequence of the first single stranded nucleic acid and the complement of the sequence of the second single stranded nucleic acid; or,
comparing the sequence of the second single stranded nucleic acid and the complement of the sequence of the first single stranded nucleic acid; or
comparing the sequence of the first single stranded nucleic acid and the complement of the sequence of the second single stranded nucleic acid and comparing the sequence of the second single stranded nucleic acid and the complement of the sequence of the first single stranded nucleic acid.

34.-37. (canceled)

38. The method of claim 1, wherein associating the sequence of the first single stranded nucleic acid with the sequence of the second single stranded nucleic acid is performed during basecalling.

39.-41. (canceled)

42. The method of claim 2, wherein the double stranded nucleic acid is not covalently attached to the substrate or a molecule immobilized thereon.

43. The method of claim 2, wherein only one strand of the double stranded nucleic acid is covalently attached to the substrate or a molecule immobilized thereon.

44. The method of claim 4, wherein the double stranded nucleic acid comprises a single-stranded region.

45. The method of claim 44, wherein the single-stranded region comprises a member selected from the group consisting of a loop, a bulge, or a partially melted the double stranded nucleic acid. 46 (New) The method of claim 6, wherein the double stranded nucleic acid is cell-free DNA or generated by fragmenting genomic DNA.
